SEQUENCE LISTING (134 sequences)

SEQ ID NO: 1 - DNA, 183 bp, Brassica oleracea
tttcaagaactggaagcttgctcccgtaaagaacgttttgaaggaccatgtgtcgatcccagaaacgagtactgcgcagcattatttaaggaatttttaaatgagaacaccgcttttaattgcacgtgtcgaactttagtttcacgtgcaaattgtcgttgtcaactagcacgcaaatgttaa

SEQ ID NO: 2 - DNA, 204 bp, Brassica oleracea
ttcatattcatcgtttcaagtcatattcaagaagtggaagctaatctgatgaagcagtgcaacattgggtatcgtatgcctggaaattgtgccgagttagcccagacatgcgaaaaattttgttcgcggggtaaggaaaagaagccttcacattggaaatgtacaaatggtccaaagaatacttattcttgcgattgtaaatag

SEQ ID NO: 3 - DNA, 216 bp, Brassica oleracea
ggtcaagatgtgcaggctaatctcatgaacaagtgtaccgattacataaatttgcttggacgatgtggcggttcaggtgacgggctctgcaggagttcatatgaaagtaataagaatacgaagcctcttaattgcgaatgtaaagatgctaaaatgaagtttcagaacgataaagatgttatacgtggacgctgtcgctgtgtactgtgtaagtaa

SEQ ID NO: 4 - DNA, 165 bp, Brassica oleracea
aatctgatgtatccgtgcgacgatacctttggtatggaaggacaatgtggcggacccaagacatgcgaaaacttatattcgaagggtttggacaagaggcctccacgttgcgaatgcacaaattctggaaagaatacttattcttgtgtatgtaaattgtgttag

SEQ ID NO: 5 - DNA, 174 bp, Brassica oleracea
ggtcaagaagtggaagctaattttatgaagaagtgtgcctctagtttccctttgagtggaccatgtcggcaaacaggagtgaaaaactgcgaaaggatatataaaaagaagccttctacatgcacatgtgaagatttttttgcagacaacaatggacgatgttgttgtgtatag

SEQ ID NO: 6 - DNA, 226 bp, Brassica oleracea
gttcaagatgtggaagctaatctgatgaatcggtgcatcgatcagttaccttttcgtggaacatgtaccagttcaggaggcgaggactgcagaaaattatttgcgactgaaacgaatatgcatccttctcgttgcaaatgtatacctgattataaaaggcgatattgtcgttgtaaactatgctaagtttttttttgggtgcacaaatatgctaagttatattagc

SEQ ID NO: 7 - DNA, 171 bp, Brassica oleracea
ggtcaagaagtggaagctaatatgatcaagaagtgtcctgatcccatcaatttgagaggaaaatgtagcgaatcaggaggggttgtcgcctgcgcaaaatcatataacaggaagaatccttctggttgctcatgtattgattatgatgaaaaaggaagatgttgtttgtga

SEQ ID NO: 8 - DNA, 195 bp, Brassica oleracea
gttcaaggagtggaagctaatctcacgaagttgtgccctggtaacgtaacttcgcgtggagtatgtggcaattcaggagtccaaagctgcgtaaccgcaatctcgaggaagctgcataaggaccgtcgactttgtagttgtttatgtaaaattcatgaaggctatcgattttgtccttgtgtatgtaaatgctaa

SEQ ID NO: 9 - DNA, 234 bp, Brassica oleracea
tttcaagaagtggaagctaatatgacgaatccgtgcatctgtaaaggaacttttcgaagaagatgtggcagtcccacaaacgattactgcggaaaattattaagggatggtcaaaatgagaagtgcgcaaaatattttagggaaaatttacatgtgaaggccgcttttgactgccaatgtttatatctacatccacacggcgtttgtacttgtcaactactacgtaaatgctaa

SEQ ID NO: 10 - DNA, 201 bp, Brassica oleracea
gttcaagaagtgaaagctaatctgaggaagcggtgccccgaacactatagtttgcctggagtttgtggcaattcaggaaacgaggagtgcaaacgccgttatccatatccgataaataaaattgatctgcctactacttgcaaatgtgaaagatccaaattccataaaaggggactttgtaaatgttcaagaaattgctaa

SEQ ID NO: 11 - DNA, 195 bp, Brassica oleracea
ttcatattcatcgtttcaagtcatattcaagaactggaagctaatctgatgaagcggtgcacccgtggctttcgtaagcctggaaaatgtaccactttagaagaagagaaatgcaaaacattatatcggccgggtcaatgcaaatgttcagatagtaaaatgaatactcattcttgtgactgtaaatcgtgttag

SEQ ID NO: 12 - DNA, 198 bp, Brassica oleracea
ggtcaagaagtggaagctgacctgttgaataattgtaaggatgcattaagattgcctggactatgttattcaggaaccctaacctgcgaaggtttatatcataaagtttgcggcaggaaagctagaagttgcaactgttttaatgattttagtaatatgaatcatgttggacattgttattgtaatttgattatctag

SEQ ID NO: 13 - DNA, 209 bp, Brassica oleracea
tttcaagaggtggaagctaatatgatgcgccagttttcctgtcacagaaattttcttggagtatgtggtactcccggcgacaagtattgcgaatcattgtttaagagaagattgaacgagcagaccgcttctaagtgtatatgtgtaccaaaacataaacgcgcaagttgtacttgtcaactaggacatcaatgctaagtttttctcgc

SEQ ID NO: 14 - DNA, 171 bp, Brassica oleracea
ggtcaagaagtggacgctaattggatgaaggggtgtgacggtcgtatccgtttgcgtcaactatgtggcccatcaggaaaggaacgctgcgaaagtataaacaaaaagaagccttctaaatgcacatgcgtagattttgatgacaaaattggacgatgttgttgtttatag

SEQ ID NO: 15 - DNA, 174 bp, Brassica oleracea
ggccaagaagtggaagctaatgtgaaggaggaatgcgccccttatttcggtttaaatggactatgtagccaatcaggagagaacccctgcgcacatcaggcgaatatgaatcgttgtaagtgcacatgtggaaatcatagaggcaaaggacaatgtttttgcagagtgaaatga

SEQ ID NO: 16 - DNA, 171 bp, Brassica oleracea
tttcgagtggaagcagtgaaacggtgtgacgacacattccctggactttgtcgcaataacggaaacaaggtgtgcgaagacttatttagcaaacataggggccagaaagtatttaattgcgactgtcaactttttactgcaaaaaaacgactttgtaagtgtaaatgctag

SEQ ID NO: 17 - DNA, 186 bp, Brassica oleracea
ggacaagaattggaagctcatctgatgaaaaattgtaaggctgatttaagattgcctggaggatgtggcaattcaagaatctcttgcgaagttttatatcatgataattgccaaaggatgcctagtaaatgcaaatgttcaaatgagagtgatggtggacgctgtgtttgttatttaattatctag

SEQ ID NO: 18 - DNA, 165 bp, Artificial Sequence
  probe specific to S haplotype of Brassicaceae
tgctcccgtaaagaacgttttgaaggaccatgtgtcgatcccagaaacgagtactgcgcagcattatttaaggaatttttaaatgagaacaccgcttttaattgcacgtgtcgaactttagtttcacgtgcaaattgtcgttgtcaactagcacgcaaatgttaa

SEQ ID NO: 19 - DNA, 159 bp, Artificial Sequence
  probe specific to S haplotype of Brassicaceae
ctgatgaagcagtgcaacattgggtatcgtatgcctggaaattgtgccgagttagcccagacatgcgaaaaattttgttcgcggggtaaggaaaagaagccttcacattggaaatgtacaaatggtccaaagaatacttattcttgcgattgtaaatag

SEQ ID NO: 20 - DNA, 195 bp, Artificial Sequence
  probe specific to S haplotype of Brassicaceae
ctcatgaacaagtgtaccgattacataaatttgcttggacgatgtggcggttcaggtgacgggctctgcaggagttcatatgaaagtaataagaatacgaagcctcttaattgcgaatgtaaagatgctaaaatgaagtttcagaacgataaagatgttatacgtggacgctgtcgctgtgtactgtgtaagtaa

SEQ ID NO: 21 - DNA, 162 bp, Artificial Sequence
  probe specific to S haplotype of Brassicaceae
ctgatgtatccgtgcgacgatacctttggtatggaaggacaatgtggcggacccaagacatgcgaaaacttatattcgaagggtttggacaagaggcctccacgttgcgaatgcacaaattctggaaagaatacttattcttgtgtatgtaaattgtgttag

SEQ ID NO: 22 - DNA, 153 bp, Artificial Sequence
  probe specific to S haplotype of Brassicaceae
tttatgaagaagtgtgcctctagtttccctttgagtggaccatgtcggcaaacaggagtgaaaaactgcgaaaggatatataaaaagaagccttctacatgcacatgtgaagatttttttgcagacaacaatggacgatgttgttgtgtatag

SEQ ID NO: 23 - DNA, 205 bp, Artificial Sequence
  probe specific to S haplotype of Brassicaceae
ctgatgaatcggtgcatcgatcagttaccttttcgtggaacatgtaccagttcaggaggcgaggactgcagaaaattatttgcgactgaaacgaatatgcatccttctcgttgcaaatgtatacctgattataaaaggcgatattgtcgttgtaaactatgctaagtttttttttgggtgcacaaatatgctaagttatattagc

SEQ ID NO: 24 - DNA, 150 bp, Artificial Sequence
  probe specific to S haplotype of Brassicaceae
atgatcaagaagtgtcctgatcccatcaatttgagaggaaaatgtagcgaatcaggaggggttgtcgcctgcgcaaaatcatataacaggaagaatccttctggttgctcatgtattgattatgatgaaaaaggaagatgttgtttgtga

SEQ ID NO: 25 - DNA, 174 bp, Artificial Sequence
  probe specific to S haplotype of Brassicaceae
ctcacgaagttgtgccctggtaacgtaacttcgcgtggagtatgtggcaattcaggagtccaaagctgcgtaaccgcaatctcgaggaagctgcataaggaccgtcgactttgtagttgtttatgtaaaattcatgaaggctatcgattttgtccttgtgtatgtaaatgctaa

SEQ ID NO: 26 - DNA, 213 bp, Artificial Sequence
  probe specific to S haplotype of Brassicaceae
atgacgaatccgtgcatctgtaaaggaacttttcgaagaagatgtggcagtcccacaaacgattactgcggaaaattattaagggatggtcaaaatgagaagtgcgcaaaatattttagggaaaatttacatgtgaaggccgcttttgactgccaatgtttatatctacatccacacggcgtttgtacttgtcaactactacgtaaatgctaa

SEQ ID NO: 27 - DNA, 180 bp, Artificial Sequence
  probe specific to S haplotype of Brassicaceae
ctgaggaagcggtgccccgaacactatagtttgcctggagtttgtggcaattcaggaaacgaggagtgcaaacgccgttatccatatccgataaataaaattgatctgcctactacttgcaaatgtgaaagatccaaattccataaaaggggactttgtaaatgttcaagaaattgctaa

SEQ ID NO: 28 - DNA, 150 bp, Artificial Sequence
  probe specific to S haplotype of Brassicaceae
ctgatgaagcggtgcacccgtggctttcgtaagcctggaaaatgtaccactttagaagaagagaaatgcaaaacattatatcggccgggtcaatgcaaatgttcagatagtaaaatgaatactcattcttgtgactgtaaatcgtgttag

SEQ ID NO: 29 - DNA, 180 bp, Artificial Sequence
  probe specific to S haplotype of Brassicaceae
gacctgttgaataattgtaaggatgcattaagattgcctggactatgttattcaggaaccctaacctgcgaaggtttatatcataaagtttgcggcaggaaagctagaagttgcaactgttttaatgattttagtaatatgaatcatgttggacattgttattgtaatttgattatctag

SEQ ID NO: 30 - DNA, 188 bp, Artificial Sequence
  probe specific to S haplotype of Brassicaceae
atgatgcgccagttttcctgtcacagaaattttcttggagtatgtggtactcccggcgacaagtattgcgaatcattgtttaagagaagattgaacgagcagaccgcttctaagtgtatatgtgtaccaaaacataaacgcgcaagttgtacttgtcaactaggacatcaatgctaagtttttctcgc

SEQ ID NO: 31 - DNA, 150 bp, Artificial Sequence
  probe specific to S haplotype of Brassicaceae
tggatgaaggggtgtgacggtcgtatccgtttgcgtcaactatgtggcccatcaggaaaggaacgctgcgaaagtataaacaaaaagaagccttctaaatgcacatgcgtagattttgatgacaaaattggacgatgttgttgtttatag

SEQ ID NO: 32 - DNA, 153 bp, Artificial Sequence
  probe specific to S haplotype of Brassicaceae
gtgaaggaggaatgcgccccttatttcggtttaaatggactatgtagccaatcaggagagaacccctgcgcacatcaggcgaatatgaatcgttgtaagtgcacatgtggaaatcatagaggcaaaggacaatgtttttgcagagtgaaatga

SEQ ID NO: 33 - DNA, 156 bp, Artificial Sequence
  probe specific to S haplotype of Brassicaceae
gtgaaacggtgtgacgacacattccctggactttgtcgcaataacggaaacaaggtgtgcgaagacttatttagcaaacataggggccagaaagtatttaattgcgactgtcaactttttactgcaaaaaaacgactttgtaagtgtaaatgctag

SEQ ID NO: 34 - DNA, 165 bp, Artificial Sequence
  probe specific to S haplotype of Brassicaceae
ctgatgaaaaattgtaaggctgatttaagattgcctggaggatgtggcaattcaagaatctcttgcgaagttttatatcatgataattgccaaaggatgcctagtaaatgcaaatgttcaaatgagagtgatggtggacgctgtgtttgttatttaattatctag

SEQ ID NO: 35 - DNA, 14 bp, Artificial Sequence
  primer
aagtggaagctaat

SEQ ID NO: 36 - DNA, 12 bp, Artificial Sequence
  primer
gaagtggaagct

SEQ ID NO: 37 - DNA, 18 bp, Artificial Sequence
  primer
ttgcctggacgttgtcgc

SEQ ID NO: 38 - DNA, 16 bp, Artificial Sequence
  primer
ttgtagttgtcaacta

SEQ ID NO: 39 - DNA, 16 bp, Artificial Sequence
  primer
tggacgatgtgattgt

SEQ ID NO: 40 - DNA, 14 bp, Artificial Sequence
  primer
ttgtgaatgtaaat

SEQ ID NO: 41 - DNA, 22 bp, Artificial Sequence
  primer
gaagacgaagcctcttaattgc

SEQ ID NO: 42 - DNA, 16 bp, Artificial Sequence
  primer
aaaggacgatgttgtt

SEQ ID NO: 43 - DNA, 17 bp, Artificial Sequence
  primer
ataaaaggggacattgt

SEQ ID NO: 44 - DNA, 29 bp, Artificial Sequence
  primer
atgaaatctgctatttatgctttattatg

SEQ ID NO: 45 - DNA, 22 bp, Artificial Sequence
  primer
ttcatattcatcgtttcaagtc

SEQ ID NO: 46 - DNA, 18 bp, Artificial Sequence
  primer
actggaagaattcgcggc

SEQ ID NO: 47 - DNA, 1322 bp, Brassica oleracea
acctccttcttgctcgtcttcgttgtcatgattctatttcatcctgccttttctatctatatcaacactttgtcggctacagaatctcttacaatctcaagcaacagaacacttgtatctcccggtaatgtcttcgagctaggcttcttcagaaccaactcaagttctcgttggtatctcgggatatggtacaagaaattgtccgggagagcatacgtatgggttgccaacagagataaccctctctccaattccattggaaccctcaaaatctccaacatgaacctcgtcctcattgatcagtccactaaatctgtttggtcaacgaatcttacgagagggaatgagagattgccggtggtggcagagcttctggctaacggaaacttcgtgatgcgagactccaataacaacaacgcaagtgcattcttgtggcaaagtttcgattaccctacagatactttgcttccagagatgaaactgggttacgacctcagaacagggcggaacaggttccttacatcatggagaagttcagatgatccgtcaaccggggatttctcgtacaagctcgaactccgaaatattcctgagttttatctattgcaaggcgactttccagagcatcggagtggtccatggaatggaatccaatttagtggcataccagaggaccaaaagttgagttacatggtgtacaatttcacagagaatagtgaggaggtcgcttatacgttcctaatgaccaacaacagcttctactcgagattgataataagttccgaagggtattttcggcgactgacgtgggctccgtcatcagtgatatggaacgtgttctggtcttctcctaatcaccagtgcgatatgtacaggatgtgtgggccttactcttactgtgacgtgaacacacaaccgatttgtaactgtatccaagggttcaatcccgggaatgtgcagcagtgggctttgagaatcccaataagtgggtgtaaaaggaggacgccgctgagctgcaatggagatggttttaccaggatgaagaatatgaagtttccagacactaggatggcgactgtcgacaggagtattggtgtgaaagaatgtaagaaaaggtgccttagcgattgtaactgtaccgcttttgcaaatgcggatatccgaaatggtgggacaggttgtgtgatttggaccggacagcttgaggatatccgaaattatgctgtcggtggtcaaaatctttatgttagattggctgcagctgatcttgttaagaagaccacagttaagaagagtaacgcgaatgggaaaatcataagttt

SEQ ID NO: 48 - DNA, 1310 bp, Brassica oleracea
acctccttcttgctcgtcttcgttgtcatgattctgtttcgtcctacactttcgatctatttcaacactttgtcgtccacagaatctcttacaatctcaaacaacagaacacttgtatcacctggtgatgtcttcgagctcggtttcttcaaaaccacttcaagttctcgttggtatctcgggatatggtacaagaaattgcccgggagaacctatgtatgggttgccaacagagataacccactctccaattccattggaaccctcaaaatctccaacatgaacctggtcctacttgatcactcgaataaatctgtttggtcgacgaatcatactagaggaaatgagagatctctggtggtggcagagcttctcgctaatggaaacttcttggtgcgagactcaaataacaacgacgcatatggattcttgtggcaaagtttcgattaccctacagatactttgcttccagagatgaaactgggttacgacctcaaaatagggctgaacaggtctcttacatcatggagaagttcagatgatccgtcaagtggggatttctcgtacaagctcgaaggttcgagaaggcttcctgagttttatctaatgcaaggcgacgttcgagagcatcggagtggtccatggaatggaatccaatttagtgggataccagaggaccaaaagttaagttacatgatgtacaatttcacagataatagtgaggaggtcgcttatacattcctaatgaccaacaacagcttctactcgagattaaaattaagttccgaagggtatttggagcgactgacgtgggctccgtcatcagggatatggaacgtgttctggtcttctccgaaccaccagtgcgatatgtacaggatgtgtggaacttactcttactgtgacgtgaacacatcaccgtcatgtaactgtatcccagggttcaatcccaagaaccggcagcagtgggatctgagaatcccaataagtgggtgtataagaaggacgcggcttggctgcagtggagatggttttaccaggatgaagaatatgaagttgccagacactacgatggcgattgtcgaccgcagtataagtgtgaaagaatgtgagaagaggtgtcttagcgattgtaattgtaccgcgtttgcaaatgcggatatccggaatcgtgggacgggttgtgtgatttggactggagagcttgaggatatgcggaattatgctgagggtggtcaagatctttatgtcagattggctgccgctgatcttgttaaaaagagaaacgcgaattggaaaatcataagttt

SEQ ID NO: 49 - DNA, 1276 bp, Brassica oleracea
acctccatcttgctcgtctttgttgtcatgattctatttcatcctgccttttcgatctatatcaacactttgtcgtctgcagactctcttacaatctcaagcaacagaacacttgtatctcccggtaatatcttcgagctcggcttcttcagaaccaactcaagttctcgttggtatctcgggatatggtacaagcaattgtccgagagaacctatgtatgggttgccaacagagataaccctctctccaattccattggaaccctcaaaatttccgatatgaacctcctcctccttgatcactctaataaatctgtttggtcgacgaatcttaccagaggaaatgagagatcatctctggtggtggcagagcttctcgctaatggaaacttcgtgatgcgagactccaataacaacgacgcaggtggattcctgtggcaaagtttcgattaccctacagatactctgcttccagagatgaaactgggttacgacctcaaaaagggactgaacagattccttacgtcgtggagaagttcagaggatccctcaagcggggaaatctcgtacaagctcgaaatgagaaggcttcctgagttttatttatggaatgaggactttccaatgcatcggagtggtccatggaatggaatcgaatttattggaataccagaggaccaaaagtcgagctacatggcgtacaatttcacagagaatagtgaaggagtcgcttatacattccgaatgaccaacaacagcttgtactcgagattgacagtaagttcagaagggaattttgagcgactgacgtggaatccgttattagggatgtggaacgtgttctggtcttctccagtggacgcccagtgcgatatgtacaggacgtgtgggccttactcttactgtgacgtgaatacatcgccggtttgtaactgtatccaagggttcaatccctcgaatgtgcagctgtgggatctgagagacggggcaggtgggtgcataaggaggacgcggcttagctgcagtggagatggttttaccaggatgaagaatatgaagttgccagaaactacgatggcgactgtcgaccggagttttggtctgaaagaatgtaagaagagatgccttagcgactgtaactgtaccgcgtttgcaaatgtggatatccggaatggtgggacaggttgtgtgttttggacaggacatctcgaggatatgcggaattacgctgctgacggtcaagatctttatgtcaaagtggctgcggctgatctcg

SEQ ID NO: 50 - DNA, 1331 bp, Brassica oleracea
accttctccttcttgctcgtcttcgttttcttgattctatttcatcctgccctttcgatctatttcaacattttgtcgtctacagaaactcttaccatctcagacaacagaacacttgtatctcccggtgatgtcttcgagctcggtttcttcaaaatcacctcaagttctcgttggtatctcgggatatggtacaagaaactctacttcggaagcatcaaaacctatgtatgggttgccaatagagatagccctctctccaatgccattggaatcctcaaaatctcaggcaataatcttttcatccttgatcactccaataaatctgtttggtcgacgaatctaactagaggaaatgagagatctccggtggtggcagagcttctcgcaaatggaaacttcgtgatgcgagactccaataacaacgacgcaagtggattcttgtggcaaagtttcgattaccctacagatactttgcttccagagatgaaactgggttacgatctcaaaaaagggctgaacaggttacttacatcatggagaagttcggatgatccttcaagcggggaaatctcgtaccaattagacactcaaaggggaatgcctgagttctatctattgataaacggctcacgataccaccggagcggtccatggaatggagtccaatttaatggcataccagaggaccaaaagttgagttatatggtgtacaattacatagagaatgatgaggaggttgcttattcatttcgaatgaccaacaacagcatctactcgagattgacaataagtttcgaagggtttttggagcgatatacgtggaccccgacatcgattgcatggaacttgttctggtcttcaccagtggatatccggtgcgatgtgtacatggcttgtgggcctgacgcttactgtaacttgaacacatcaccattgtgtaactgtatccaagggttcaagcgctccaatgagcagcagtgggatgtgagagacgggtcaagtgggtgtataagggagacgcggctgagctgcagtggtgatggttttaccaggatgaagaagatgaagttgccagaaactacaacggcgattgtcgacaggagtattggtgtgaaagaatgtgagaagaggtgccttagcgattgtaattgtacagcgtttgcaaatgcggatatccggaatggtgggacgggttgtgtgatttggaccacagggcttgaggatatccggacttactttgctgctgatcttggtcaagatctttatgtcagattggctgctgctgaccttgttaaaaagagtaacgcgaatgggaaaatcatatcttt

SEQ ID NO: 51 - DNA, 483 bp, Brassica oleracea
ttcagatgatccgtcaagcggggattactcgtacaagctcgaaccccgaaggcttcctgagttttatctactgcaaggagacgtccgagagcatcggagtggtccatggaacggaatccgatttagtgggatactagaggaccaaaagttgagttacatggtgtacaatttcacagagaatagtgaggaggtcgcttatacattccgaatgaccaacaacagtttctactcgagattgacactaagctccacagggtattttgagcgactgacgtgggctccgtcatcagtgatatggaacgtcttctggtcttctccagcaaacccccagtgcgatatgtacaggatgtgtgggccttactcttactgtgacgtgaacacatcaccatcgtgtaactgtatacaagggttcgatcccaggaatttgcagcagtgggctctgagaatctcattaagggggtgtaaaaggaggacgctgctgag

SEQ ID NO: 52 - DNA, 1273 bp, Brassica oleracea
acctttttcttgctcgtcttcgttgtcatgattctatttcatcctgccctttcgatgtatttcaacactttgtcctctacagaatctcttacaatttcaaacaacagaacacttgcatctcccggtgatgtcttccagctcggtttcttcagaaccaactcaagttctccttggtatctcgggatatggtacaagcaattgtccgacagaacctatgtatgggttgccaacagagatagccctctttccaacgccattggaatcctcaaaatctctggcaataatcttgtcatccttgatcactccaataaatctgtttggtcaacgaatcttacaagaggaaatgagagatctccggtggtggcagagcttctcgctaacggaaacttcgtggtgcgagactccaataacaacggtgcaagtggattcttgtggcaaagtttcgattaccccacagatactttgcttccagagatgaaactgggctacgacctcaaaacagggctaaacaggtttcttacatcatggaagagttgggatgatccatcaagcggggatttcttgtacgagctcgaaacccgaaggcttcctgaattttatctaacgattgggatctttcgagtgcatcggagtggtccatggaatggaatccgatttagtggcataccagatgaccaaaagttgagttacttggtgtacaattttacagagaatagtgaagaagttacttatacattccgaatgaccaacaacagcatctactcgagattgatagtaagtttctcagggtatattcagcgacagacgtggaatccgacattagggatgtggagcgtattctggtcttttccattcgactcacagtgcgattcgtacagagcgtgtgggcctaacgcttactgtgacgtgaatacatcaccgttttgtaactgtatccaagggttcattccctcgaatgtggtgcagtgggatcagagagtctgggcaggtgggtgtataaggaggacgcgtcttagctgcagtagagatggttttaccaggatgaagaatatgaagttgccagaaactacgatggctattgtcgaccgcagtactggtgtaaaagaatgtaagaagaggtgccttagcgattgtaactgtaccgcgtttgcaaatgcggatatccggaatggtgggacgggctgtgtgatttggaccggacagtttcacgatatgcggaattacggtgttgacggtcaaaatctttatgtccgactggctgctgctgatctcg

SEQ ID NO: 53 - DNA, 1307 bp, Brassica oleracea
accttcttgctcgtctttttcgtcttgattctatttcgacctgccttttcgatcaacgctttgtcggctacagaatctcttacaatctcaaacaacagaacactagtatctcccggtgatgtcttcgagctcggtttctttataaccaactcaagttctcgttggtatctcgggatatggtacaagaaattgtccgagagaacctatgtatgggttgccaacagagatagccctctctccaatgccattggaaccctcaaaatctcagacaataatcttgtactccttgatcactccaataaatctgtttggtcaacgaatctcactagaggaaatgagagatctccggtggtggcagagcttctcgcaaatggaaacttcgtgatgcgagactccaataacaacgacgcaagtggattcttgtggcaaagtttcgattcccctacagatactttgcttccagagatgaagctgggttacgacctcaaaacagggctgaacaggttccttacaccatggagaagttcagatgatccgtcaagcggggatttcttgtacgagctcgaagcccggaggcttcctgaattttatctatctagtgggatctttcgattgtatcggagcggtccatggaatggaatccgatttagtggcataccagatgaccaaaagttgagttacatggtgtacaatttcacagagaatagtgaagaagtcgcttatacattccgaatgaccaataacagcatttactcgaaattgacagtaagtgtctcagggaagtttgagcgacagacgtggaatccgacattagggatgtggaacgtgttctggtctttcccgtcggactcacagtgcgatacgtacaggatttgtgggccttattcttattgtgacgtgagcacatcaccgatttgtaactgtatccaagggttcaatccctcgaatgtgcagcagtgggatcagagatcctggtcaggtgggtgtataaggaggacgcaacttagctgcagtggaaatggttttgcaaggatgaagaatatgaagttgccagaaattaggatggctattgtcgaccgcagtattggtattggtgtgaaagaatgtgagaaaaggtgccttagcgactgtaattgtaccgcgtttgccaatgtggatattcggaatggtgggacgggttgtgtgatttggaccggacggcttgacgatatgcggaattacgcttctgatggtcaagatctttatgtcaaactggctgcagcagatatcgtaaagaagagaaaccccaatgggaaaatcataagttt

SEQ ID NO: 54 - DNA, 1292 bp, Brassica oleracea
accttctccttcttgctcgtcttcgttgtcatgattttatttcatcctgccctttcgatgtatttcaacactttgtcgtctacagaatctctttcaatctctaacaacagaacactattatctcccggtaatgttttcgagctcggcttcttcagaaccaattctcgttggtatctcgggatgtggtacaaggaattgtccgagaaaacctatgtatgggttgccaacagagataaccctctcgccaatgccattggaaccctcaaaatctctggcaataatcttgtcgtccttgatcactccaataaatctgtttggtcgacgaatcttactagagaaaatgagagatctccggtggtggcagagcttctggctaatggaaacttcgtgatgcgagactcaagtggatttttgtggcaaagtttcgattaccctacagatactttgcttccagaaatgaaactgggttacgacctcaaaacagggttgaacaggttccttatatcatggagaagtttagatgatccgtcaagcgggaatttctcgtacagcctcgaaaaacgagagcttcctgagttttatctatataaaggcgactttcgagtgcatcggagtggtccatggaatggaatcgcatttagtggcataccagaggaccaacagttgagttacatggtgtacaatttcacagagaatagggatgaggccgcttatacattccgaatgacgaacagcagcatctactcgaaattgacaataaattcggaagggagatttcagcgactgacgtggactccatcatcaggcgcgtggaacgtgttctggtcttctcctgtgaaccccgagtgcgatttgtacatgatttgtgggccttacgcttactgtgacttgaacacatcaccgtcgtgtaactgtatccaagggttcaatcccggggatgtgcagcagtgggatctgagagactggacaagtgggtgcataaggaggacacgcctgagatgcagtggcgatggttttacaaggatgaagaatatgaagttgccagaaactacgatggctattgtcgaccgcagtattggtatgaaagaatgtaagaagagatgccttagcgattgtaattgtacagcttttgcaaatgcggatgtccggaatggtgggacgggttgtgcgatttggaccgcacagcttgacgatgtccggaattacggtgctgacggtcaagatctttatgtcagattggctgctgctgatctcgttaagaggagaaacgcgaatgggaaaatcataagttt

SEQ ID NO: 55 - DNA, 1316 bp, Brassica oleracea
accttccccttcttcctcgtcctcgctgtcttgattctattttatcctgcccattcaatgtatttcaacactttgttgtctagagaatctcttacaatcacaagcaacagaacacttgtatctcccggtgatgtcttcgagctcggtttcttcaagaccacttcaagttctcgttggtatctcgggatatggtacaagaaactctccgaaagaacctacgtatgggttgccaacagagatagccctctctcaaatgccgttggaacccttaaaatctccaacatgaacctggtcctccttgatctctctaataaatctgtttggtcgacgaatcttacgagaggaaatgagagatcaccggtggtggcagagcttctcgctaatggaaactttgtgatacgatactccaataaaaactacgcaactggattcttgtggcaaagtttcgattaccctacagatactttgcttccagatatgaaattgggttacgactttaaaaaagggctgaacagattcctaacatcatggagaaattcagatgatccatcaagcggggaaatctcgtacaaactagacactcaaacgggaatgcctgagttctatctattgcaaacgggcgtacaagtgcatcggagcggtccatggaatggagtccgatttagtggcataccaggggaccaagagttgagttacatggtgtacaatttcacagagaatactgaggaggttgcttatacatttcgaatgactgacaacagcatctactcgagattgaaagtaagttccgaagggtttttggagcgactgacgtggaccccgaactcaactacatggaacttgttctggtatttaccattggaaaaccagtgcgatatgtacatgatttgtgggcgttacgcttactgtgacgtgaacacatcacccttgtgtaactgtatccaagggttcataccctggaataagcagcagtgggatcagagagacccgtcaggtgggtgtaaaaggaggacgaggcttagctgcaatggagatggttttacaaggatgaagaaaatgaagttgccagaaactacgatggcaactgtcgaccgcagtattggtgtgaaagaatgtgagaagaggtgccttagcgactgtaactgtaccgcgtttgcaaaggcggatatccggaatggtgggacgggttgtgtgatttggaccggagctctcgaggatataagaaattactatgctgacggtcaagatctttatgtcagattggctgccgctgatcttgttaaaaagagaaacgcgaattggaaaatcataagtgt

SEQ ID NO: 56 - DNA, 1537 bp, Brassica oleracea
acctccttgctcgtcttcgttgtcatgattctatttcgttctgccctttcgatctatatcaacactttgtcctctacagaatctcttacaatctcaaacaacagaacacttgtatctcccggtggtgttttcgagctcggtttcttcactctcggatcaagttctcgttggtatctcgggatatggtacaagaaactgccctatataacctatgtatgggttgccaacagagataaccctctttccaactccactggaaccctcaaaatctcaggcaataatcttttcctccttggtgactccaataaatctgtttggtcgacgaatctaactagaggaaatgagagatctccggtggtggcagagcttctcgctaatggaaacttcgtgatgcgagactccaataacaacgacgcaagtggattcttatggcaaagtttcgatttccctacagatactttgcttccagagatgaaactgggttactacctcaaaacagggctgaacagattccttacatcatcgagaagtttcgacgatccatcaagcggggattactcgtacaagctcgaaccccgaaggcttcctgagttttatctattgctaggcgacgttcgtgagcatcggagcggtccatggaatggaatccaatttagtggaataccagaggaccaaaagttgagttacatggtgtacaatttcacagagaatagtgaggaggttgcttatacatttcgaatgaccaacaatagcttctactcgagattgacaataaattctgaagggtatttggagcgacagacgtgggctccgtcatcagtggtatggaacgtcttctggtcttctcctaaccatcagtgcgatatgtacaggatgtgtgggccttactcttactgtgacgtgaacacatcaccgtcatgtaactgtatccaagggttcaaacccgggaatgtgcagcagtgggctctgagaaaccaaataagtgggtgtaaaaggaggacgcggctgagctgcaatggagatggttttaccaggatgaagaatatgaagttgccagacactacgatggcgattgtcgaccgcagtatgagtgtgaaagaatgtgagaagaggtgtcttagcgattgtaattgtaccgcgtttgcaaatgcggatatccggaatcgtgggacgggttgtgtgatttggactggagagcttgaggatatgcggaattatgctgagagtggtcaagatctttatgtcagattggctgccgctgatcttgttaaaaagagagacgcgaattggaaaatcataattgttggagttagtgttgttctgcttctgcttctgcttcttctgatcatgttctgcctttggaaaaggaaacaaaatcgagcaaaagcaatggcaacatctattgtcaatcaacagagaaaccaaaatgtacttatgaacgggatgacacaatcaaacaagagacagttgtctagagagaacaaaactgaggaattcgaacttccattgatagagttggaagctgttgtcaaagcca

SEQ ID NO: 57 - DNA, 1316 bp, Brassica oleracea
accttctccttcttgctcgtcttcgctgtcttgattctgtttcatcctgccctttcgatctatttcaacattttgtcgtctacagaatctcttacaatctcaaccaacagaacacttgtatctcccggtaatgtcttcgagctcggcttcttcagaaccaactcaagttctcgttggtatctcgggatatggtacaagaaaatctcggaaagaacctatgtatgggttgccaacagagataggcctctctccagtgccgttggaacccttaaaatctctggctataatcttgtcctccgaggccactccaataaatcagtttggtcgacgaatcttacaagaggaaatgagagatcaccggtggtggcagagcttctcgctaatggaaacttcgtgatgcgagactccaataacaacaacgcaagtcaattcttgtggcaaagtttcgattaccctacagatactttgcttccagagatgaaactgggttacgacctcaaaacagggttgaacaggttccttacatcatggagaacttcagatgatccgtcaagcggggattacttgtacaagctcgaaccccgaaaacttccggagttttatctatggaatgaggactttccaatgcatcggagcggtccatggaatggagtcagatttagtggtataccagaggaccaaaagttgagctacttggtgtataatttcacagagaatagtgaagaggtggcttacacattccgaatgaccaacaacagcttttactcgagattgacagtaagttcctcagggtattttgagcgactgacgtggaatccgtcattagggatatggaacgtgttctggtcttctccagtggacttccattgcgacttgtacgtgagttgtgggccttactcttactgtgacgtgaacacatcacctgtgtgtaactgtatccaagggttcaatccctggaatatgcaggagtggaatctgagagtaccggcaggtgggtgtataaggaggaccaagcttagctgcagtggagatggttttaccaggatgaagaatatgaagttgccagaaacaacgatggctattgtcgacaggagtattggtttgaaagaatgtgagaagaagtgccttagtgactgtaattgtaccgcgtttgcaaatgcggatattcggaatcgtgggacgggttgtgtcatttggaccggacggcttgccgatatgcggaatttcgttcctgatcacggtcaagatctttatgtcagattggctgctgatgatctcgttaagaagagaaacggtaatgggaaaatcataagttt

SEQ ID NO: 58 - DNA, 1279 bp, Brassica oleracea
accttgtccttcttgctcgtcttcgttgtcatgtttctagttcatcctgcccttgcgatctatatcaacactttgtcgtctacggaatctcttacgatctcaagcaacagaacacttgtatctcccggtaatgacttcgagctcggcttcttcagaaccacctcaagttctcgttggtacctcgggatatggtacaagaaagtgtccgacagaacctatgtatgggttgccaacagagataaccctctcctcagttccattggaaccctcaaaatctcaggcaataatcttgtcatacttggtcactccaataaatctgtttggtcgacaaatgtaactagaggaaatgagagatcaccggtggtggcagagcttctcgctaatggaaacttcgtgatgcgagactccaataacaacgacgcaagtgggttcttgtggcaaagtttcaatttcccgacagatactttgcttccagagatgaaactgggttacgacctcaaaacagggctgaacaggttccttacatcatggagaagtttagatgatccgtcaagcggggattactcgtacaagctccaagcccgaagttatcctgagttttatctaattaaaaaaaaagtctttattgggcatcggagtggtccatggaatggaatccgatttagtgggataccagaggaccaaaagttgagttacatggtgtacaatttcacagagaatagagaggagatcgcttatacatttcgaatgaccaacaacagcttctactcgcgattgacaataagttccgaagggtattttgagcgtctgacgtggactctgtcatcaaatatgtggagcgttttctggtcttctccagtggacctccagtgcgatgtgtacaagtcttgtgggccttactcttactgtgacgtaaacacatcaccggtgtgtaactgtgtccaagggttctatcccaagaaccagcagcagtgggatgtgagagtcgcttccagtgggtgtataaggaggacgcgtcttagctgcaatggtgatggttttacaaggatgaagaacatgaagttgccagaaactacgatggctattgtcgaccgcagtattggtgagaaagaatgtgagaagaggtgccttagcgattgtaattgtaccgcgtttgcaaatgcggatatccggaatggtgggacgggttgtgtcatttggaccggagagcttgaggatatcaggaattacgctgctgacggtcaagatctttatgtcagattggctgcagctgaccttg

SEQ ID NO: 59 - DNA, 1276 bp, Brassica oleracea
accttatccttcttgctagtctttttcgtcatgattctatttcgtcctgccctttcgatgtatttcaacactttgtcgtctacagaatctcttacaatttcaaacaacagaacacttgtatctcccggtgatgtcttcgagcttggtttcttcagaaccacctcaagttctcgttggtatctcgggatatggtacaagaaactgccctttagaacctatgtatgggttgccaacagagataaccctctctccaactccattggaaccctcaaaatctcaggcaataatcttgtcatccttggtcactccaataaatctgtctggtcgacgaatcttacaagaggaagtgagagatctacggtggtggcagagcttctcgctaatggaaacttcgtgatgcgagactccaataacaacgacgcaagtgcattcttgtggcaaagtttcgatttccctacagatacgttgcttccagagatgaaactgggttacgacctcaaaacagggctgaataggttccttacatcatggagaagttcagatgatccatcaagcggggagttcttgtacgagctcgaaaccggaagacttcctgaattttatctatcgaaggggatctttccagcgtatcggagtggtccatggaatggaatccgatttagtggcataccagatgaccaaaagttgagttacctggtggacaatttcacagataatagtgaagaagtcacttatacattccgaatgaccaacaacagcatctactcgaagttgacagtaagtttctcagggtattttgagcgacagacgtggaatccgtcattagggatgtggaacatgttctgggcttttccaatggcctcacagtgcgatacgtacaggaggtgtgggccttactcttactgtgacgtgagcacatcaccgatttgtaactgtatccaagggttcaatccctcgaatgtgcagcagtgggatcagagatcctggtcaggtgggtgtataaggaggacgaggcttagctgcagtggagatggttttaccaggatgaagaatatgaagttgccagaaactacgatggctattgttgaccggagtattggtgtgaaagaatgtaagaaaaggtgccttagcgattgtaattgtactgcgtttgcaaatgcggatgtccagaatggtggaactggttgtataattcggacaggagagctcgaggatatccggaattacgctgctgacagtcaagatctttatgtcagactggctgctgctgatctcg

SEQ ID NO: 60 - DNA, 1319 bp, Brassica oleracea
accttctccttcttgctcgtcttcgttgtcttgattctatttcatcctgccctttctatctactttaacactttgtcgtctacagatactcttaccatctcaagcaacagaacacttgtatctcccagtgatgtcttcgagctcggtttcttcaaaacctcttcaagttctcgttggtacctcgggatatggtacaagacagtctcctacagaacctacgtatgggtggccaacagagatagccctcttttcagtgccactggaaccctcaaaatttcaggcaataatcttgtcctccttggcaaatccaataaatctgtttggtcgacgaatcttacgagaagaattgagagttcaccggtgatggcagagcttctcgctaatggaaactttgtgatacgagactccaataacaacgacgcaagtggattcttgtggcaaagtttcgatttcccgacggatactttgcttccagagatgaaactgggttacgaccgcaaaaaagggctgaacagattccttacatcatggagaaattcagatgatccctcaagcggggaaatctcgtaccaactagacgctactccaagcggaatgtatgagttctatctattgaaaagcggctcacgagcccaccggagcggtccatggaatggagtccgatttagtggcataccaggggatcaagagttgagttacatggtgtacaatttcacagagaatagtgaggaggtttcttattcatttagaatgaccaacaacagcatctactcgatattgaaagtaagttccgatggggttttggagcgactgacctggaccccgaactcaattggatggaacttgttctggtatttaccattggaaaaccagtgcgatgtgtacatggtttgtgggcgttactcttactgtgacgtgaacacatcacccttgtgtaactgtatccaagggttcaatcgctcgaatgaggagcggtgggatctgaaagactggtcaagcgggtgtatgaggaggacgcagctgagctgcagtggagatggttttaccaggatgacgaagatgaagttgccagagacaaagatggcaattgtcgacaggagtattggtgtgaaagaatgtaggaagaggtgccttagcgattgtaattgtacagcgtttgcaaatgcggatatccggaatggtgggacgggttgtgtgatttggaccggacagctttacgatatccggaattactatgctgacggtcaagatctttatgtcagattggctgctgctgatcttgttaaaaagagaaacgcgaatgggaaaatcataacttt

SEQ ID NO: 61 - DNA, 1273 bp, Brassica oleracea
accttcttgctgttcttcgttgtcatgattctatttctgcctgccctttcgatctatatcaacactttgtcgtctacagaatctcttacaatctcaagcaacagaacacttgtatctcccggtgatgtcttcgagctcggcttcttcagaaccaattctcgttggtatctcgggatgtggtacaagaaactgccctatagaacctatgtatgggttgccaacagagataaccctctctccaactccattggaaccctcaaaatctcaggcaataatcttgtcatccttggtcactccaataaatctgtttggtcgacgaatcttacaagaggaagtgagagatctccggtggtggcagagcttctcggtaacggaaacttcgtgatacgatactccaataacaacaacgcaagtggattcttgtggcaaagtttcgatttccctacagatactttgcttccagagatgaaactgggttacgacctcaaaaaagggtttaacaggttccttatatcatggagaagttcagatgatccttcaagcgggaattactcgtacaagctcgaaacccgaaggcttcctgagttttatctatcgagtggagtttttcgattgcatcgtagtggtccgtggaatggaatccaaattagtggcataccagaggaccaaaatttgcattacatggtgtacaatttcatagagaatagtgaagaggtcgcttatacattccgaatgaccaacaacagcatctactcgagactgacactaggtttctcaggggactttcagcgactgacgtggaatccgtcaatagggatatggatcttgttctggtcttctccagtggacccccagtgcgatacatacgtaatgtgtgggcctaacgcttactgtgacgtgaacacatcaccggtatgtaactgtatccaagggttcaatccctggaatgtgcagctgtgggatcagagagtctgggcaggtgggtgtataagaaggacgcagcttagctgcagtggagatggttttaccaggatgaagaagatgaagttgccagaaactacgatggcaattgtcgaccgcagtattggtgtgaaagaatgtgagaagaggtgccttagcgattgtaattgtaccgcttttgcaaatgcggatatccggaatggtgggacgggttgtgtgatttggaccgaacagcttgatgatatgcggaattacggtactggcgctactgacggtcaagatctttatgtcagattggctgcagctgatatag

SEQ ID NO: 62 - DNA, 1284 bp, Brassica oleracea
acctccttcttgctcgttttcattgtcatgattctatttcatcctgccctttcgatctacttcaacactttgtcgtctacagaaactcttaccatttcaagcaacagaacacttgtatctcccagtgatgtcttcgagctcggcttcttcagaaccaactcaagctctggttggtatctcgggatatggtacaagaaagtctcctacagaacctacgtatgggtggccaacagagatagccctctcttcaatgccattggaaccctcaaaatctctggcaataatcttgttctccgaggccaatccaataaatctgtttggtcgaccgatcttacgagaggaaatgagagatttccggtggtggcagagcttctcgctaatggaaacttcgtgatacgatactccaataaaaatgacgcaagtggattcttgtggcaaagtttcgattaccctacagatactttgcttccagagatgaaactgggttacgacctcaaaacagagcagaacaggttccttacatcatggagaaattcagatgatccctcaagcggggaaatctcgtactttctagacactgagagcggaatgcctgagttctatctattgaaaagcggcttacgagcctaccggagtggtccatggaatggagtccgatttagtggcataccaggggaccaatatttgagttacatggtgttcaatttcacagagaatagtgaggaggttgcttatacatttcgaatgaccacccacagcatctactcgagattgaaaataagttctgaagggtttttggagcgattgacgtggaccccgaactcaattcaatggaacttgttctggtatttaccagtggaaaaccagtgcgatgtgtacatggtttgtggggtttactcttactgtgacgagaacacatcaccggtgtgtaactgtatccaagggttcatgcccttgaatgagcagcgatgggatctgagagactggtcaagcgggtgcacaaggaggacgcggcttagctgcagtggtgatggttttacaaggatgaggaagatgaagttgccagagactaagatggcgaatgtctacaggagtatcggtgtgaaagaatgtgagaagaggtgccttagcgattgtaattgtaccgcatttgcaaatgcggatatccggaatggtgggaccggttgtgtgatttggaccggacggcttgacgatatccggaattactatgctgacggtcaagatctttatgtcagattggctgctgctgatcttgtaaattga

SEQ ID NO: 63 - DNA, 1322 bp, Brassica oleracea
accttctccttcttgctcgtctttgttgtcttgattctatttcatcctgccctttcgatctattttaacattttgtcgtctacagaaacacttaccatctcaggcaacagaacacttgtatctcccggtgatgtcttcgagctcggtttcttcaaaaccaccttaagttctcgttggtatctggggatgtggtacaagaaagtctacttcaaaacctacgtatgggttgccaacagagatagccccctctccaatgccattggaaccctcaaaatctctggcagtaatcttgtcctccttgatcactctaataaatcagtttggtcgacgaatcttactagaggaaatgagagatctccggtggtggcagagcttctcgctaacggaaacttcgtgatacgatacttcagtaacaacggtgctagtggattcttgtggcaaagtttcgattaccctacagatactttgcttccagagatgaaactgggttacgaccacaaaacagggctgaacaggttgcttacatcatggagaagttcagatgatccctcgaggggggaattctcgtaccaactagacactcaaaggggaatgcctgagttctttataatgaaagaaggctcacaaggccaacggagcggtccatggaatggagtccaatttagtggcataccagaggaccgaaagttgagttacatggtgtacaatttcacagagaataatgaggaggttgcttatacgtttcgagtgaccaacaacagcttttactcgagactgaaaataagtcccgaaggggttttagagcgattgacgaggaccccgacaacagttgcatggaacgtgttctggtctgtaccagtggatacccggtgcgatgtgtacatggcttgtgggccttacgcttactgtgacatgaatacatcaccgttgtgtaactgtatccaagggttcaagcgcttcaatgagcaggaatgggaaatgagagacgggtcaagtgggtgtataagggggacgcggctgagctgcagtggtgatggttttaccaggatgaagaagatgaagttgccagacactatgatggcgattgtcgacaggagtattggtatgaaagaatgtgagaagaggtgccttagtgattgtaattgtaccgcgtttgcaaatgcggatgtccggaatggtgggacgggttgtgtgatttggaccggacagcttgacgatatgcggaattactttgctgctgatcttggtcaagacctttatgtcagattggctgccgctgatcttgttaaggagagaaatgcgaatgggaaaatcataacctt

SEQ ID NO: 64 - DNA, 1276 bp, Brassica oleracea
accttctctttcgtgctcgcctttgttgtcttgattctgtttcatcctgccctttcgatgtatttcaacactttgttgtctacagaatctcttaccatctcaggcaacagaacacttgtatctcccggtgatgtcttcgagctcggtttcttcaaaaacactttaaattctcgttggtatctcgggatatggtacaagaatctctccgacagaacctacgtatgggttgccaacagagatagctctctctccaatgccattggaacccttaaattctctggcagtaatcttgtccttcggggccgttccaataaatttgtttggtcgacgaatcttactagaggaaatgagagatctcccgtggtggcagagcttctcgctaacggaaacttcgtgatacgatactcagataacaacgatgcaagtggattcttgtggcaaagtttcgatttccctacagatactttgcttccagagatgaaactaggttactacctcaaaacagggctgaacagattccttacatcttggagaaattttgatgatccgtcaagtggagaattctcgtacaagctcgaaacccgaaggcttcctgagttttatctattgaaaaacggctcaccaggtcagcggagcggtccatggaatggagttcaatttagtggcataccagaggaccaaaagttgagttacatggtgtacaatttcacagagaatagtgaggaggttgcttatacatttcgaatgaccgacaacagcatttactcgagaattcaaataagtcccgaagggcttttggagcgactgacgtggactccgacatcagggacatggaacttgttctggtctgcaccagtggatatccagtgcgatgtgtacatgacgtgtgggccttacgcttactgtgacgtgaacacatcaccggtgtgtaactgtatccaagggttcatcccctttgatatgcagcagtgggctctgagagacgggacaggtgggtgtataaggaggacgcggctgagctgcagtagtgatggtttcaccaggatgaagaatatgaagttgccagacactaagatggcgattgtcgacaggagtattgatgtgaaagaatgtgagaagaggtgccttagcgattgtaattgtaccgcgtttgcaaatgcggatatccggaatggtgggacgggttgtgtgacttggaacagagagcttgaggatatccggagttacattagcaacggtcaagatctttatgtcagattagctgcagcggatctcg

SEQ ID NO: 65 - DNA, 1307 bp, Brassica oleracea
accttcttgctcgtcttcgttgtcatgattctatttctgcctgccctttcgatctatatcaacactttgtcgtctacagaatctcttacaatctcaagcaacagaacacttgtatctcccggtgatgtattcgagctcggtttcttcagaaccacctcaagttctccttggtacctcgggatatggtacaagcaattgtccgagagaacctatgtctgggttgccaacagaggtaaccctctccccaattccattggaagcctcaaaatctcaggcaataatcttgtcctccttggtcactccaataaatcagtttggtcaacgaatctaactagagaaaatgagagatctccggtggtggcagagcttctcgcaaatggaaacttcgtgatgcgagactccaataacaacgacgcaagtggattcttgtggcaaagtttcgattaccctacagatactttgcttccagagatgaagctgggttacgaccacaaaacagggctgaacaggttccttaactcatggagaagtttaaatgatccctcaagcgggaattactcgtacaggctcgaaacccgaaggtttcctgaattttatctatggagtggggtctttatattgtatcggagtggtccatggaatggaatccgatttagtggcatactagaagaccaaaagttgagttacatggtgtacaatttcacagagaatagtgaagaagtcgcttatacatttcgaatgaccaacaacagcatgtatacgagattgacagtaagtttctcaggggattttgaacgacagacgtggaatccgtcaatagggatgtggaacaggttctgggcttttccattggactcacagtgcgatgcgtacacagcgtgtgggccttactcttactgtgacgtaaccacatcaccgatttgtaactgtatccaagggttcaatccctcgaatgtggagcagtgggatctgagaagctggtttggtgggtgtataaggaggacgcggcttagctgcagtggtgatggttttacaaggatgaagaatatgaagttgccagaaactacgatggctattgtcgaccgcagtattggtgtgaaagaatgtaaagaaaggtgccttagcgattgtaattgtactgcgtttgcaaatgcggatatccggaatggtgggacgggttgtgtgatttggaccggagagcttatcgatatgcggaattacgttgctgacggtcaagatctttatgtcagattggctgctgctgaccttgttacgaagagaaacgcgaattggaaaatcataagttt

SEQ ID NO: 66 - DNA, 1267 bp, Brassica oleracea
accttctccttcgttgtcatgattctatttcgccctgccttttcgatctatatcaacactttgtcgtctacagaatctctcacaatctcaaacaacagaacacttgtatctcccggtgatgtcttcgagctcggtttctttagaaccaactcaagttctcgttggtatctcgggatactgtacaagcaattgtccgagagaacctatgcatgggttgccaacagagataaccctctccccaattctattggaaccctcaaaatctccaacatgaaccttgtcctccttgatcactctaataaatctgtttggtcgacgaatcttactagagtaaatgagagatcatctccggtggtggcagagcttctcgctaatggaaacttcgtgatgcgacactccaataacaacgacgcaagccaattcctgtggcaaagtttcgattaccctacagatactttgcttccagagatgaagctgggttacgacctcaaaacagggatgaacaggttccttacatcatggagaagttcagatgatccgtcaagcggcgatttctcgtacaagctcgaagcccaaaggcttcctgagttttatctatcgagcggggtctttcgattgtatcggagcggtccatggaatggagtccgatttagtggtataccagatgaccaaaagttgagttacttggtgtacaatttcacagagaatagtgaggaagtcgcttatacattccgaatgaccaacagcagcatttactcgagattgatgctaagtttctcagggtatattgagcgacagacgtggaatccatcattaaggatgtggaacgtgttctggtcttttccattggactcacagtgcgattcgtaccggatgtgtgggcctaacgcttactgtgacgtgaacacatcaccgatttgtaactgtatccaagggttcaatccctcgaatgtgcagcagtgggatcagagagtctgggcaggtgggtgtataaggaggacgcggcttagctgcagcggagatggttttaccaggatgaagaatgtgaagttgccagaaactacgatagctactgtcgaccgcagtattggtgtgaaagaatgtgagaagaggtgccttagcgattgtaattgcaccgcgtttgcgaatgcggatatccagaatggtgggatgggttgtgtgatttggaccggacggtttcacgatatgcgaaattacgctgctgacggtcaagatctttacgtcagactggctgctgctgatctcg

SEQ ID NO: 67 - DNA, 1273 bp, Brassica oleracea
accttatccttcttgctcgtcttcgttgtcatgattctatttcatcctgccctttcgatgtatttcaacactttgacgtctacggaatctcttacaatctcaaacaacagaacacttgtatctcccggtgatgtcttcgagctcggcttcttcagaaccaattctcgttggtatctcgggatatggtacaagaaactctccgagagagcctacgtatgggttgccaacagagatagccctctctccaattccattggaaccctcaaaatctctggcaataatcttgttctccgaggtaactccaataaatctgtttggtcaacgaatcttactagaagaaatgagagatctccggcggtggcagagcttctcgctaacggaaacttcgtgatacgatacttcaacaacaacaacgcaagtgaattcttgtggcaaagtttcgatttccctacagatactttgcttccagagatgaaactgggtttcgatctcaagacagggctgaacaggttccttacatcatggagaaattatgatgatccctcaagcggggaaatctcgtacaaactagacactgaaaggggattgccagagttttatctattgaaaaacggcttacgagcccaccggagcggtctatggaatggagtccaattttatggcataccagaggacctaaaattgagttacatggtgtacaactacacagagaatagtgaggaggtcgcttatacatttcgagtgaccaacaacagcatctattcgatattgaaagtaagttccggagagtttttggcgagactgactacgactccgtcatcatgggaatggagcttgttctggtattcaccagcggagccccagtgcgatgtgtacaagacttgtgggccttactcttactgtgacgtgaacacgtcaccggtgtgtaactgtatccaagggttcatgcccaggaatgtccagcagtgggagctgagaaacccgtcaggtgggtgtataaggaggacgcagctgagctgcagtggagatggttttaccaggatgaaaaagatgaacctgccagagacttcgatggcggttgttgacaggagtattggtgttaaagaatgtaagaaaaggtgccttagcgactgtaactgtaccgcgtttgcaaatgcggatatccggaatggtgggacgggttgtgtgatttggaccggagagcttgaagatatccggaattactttgatgacggtcaagatctttatgtcagattggctgccgctgatctcg

SEQ ID NO: 68 - DNA, 1316 bp, Brassica oleracea
acctccttctcggccgtctttttcttcatgattctatttcatcctgccctttcgatctatatcaacactttgtcatctagagaatctcttaaaatctcaagcaacagaacacttgtatctcccggtagtatcttcgagctcggcttcttcagaaccaattctcgttggtatctcgggatatggtacaagaaacttccctatagaacttatgtatgggttgccaacagagataaccctctttccaactccactggaaccctcaaaatctcaggcaataatcttgtcatccttggtcactccaataaatctgtttggtctacgaatcttacaagaggaagtgagagatctacggtggtggcagagcttctcgctaacggaaacttcgtgatgcgagactccaataaaaacgacgcaagtggattcttgtggcaaagtttcgatttccctacagatactttgcttccagagatgaaactgggttacgacctcaaaacagggctgaacaggttccttacatcatggagaagttcagatgatccctcaagcgggaatttctcgtacaagctcgaaaaccaaaggcttcctgagttttatctatcgagtcatggaatttttcgattgcatcgaagtggtccatggaatggaatcggatttagtggcataccagaggacgaaaagttgagttacatggtgtacaatttcacagagaatagtgaagaggttgcttatacattccgaatgaccaacaacagcatctattcgagattgacactaatttccaaaggggattttcagcgactgacgtgggatccgtcattagaaatatggaacatgttctggtcttctccagtggacccccagtgcgattcatacataatgtgtggggcgtacgcttactgtgacgtgaacacatcaccggtatgtaactgtatccaagggttcaacccccggaatatacagcggtgggatcagagagtctgggcaggtgggtgtgtaagaaggacgcggctaagctgcagtggagatggttttactaggatgaagaagatgaagttgccagaaactacgatggctattgtcgaccgcagtattggtgtgaaagaatgtaagaaaaggtgccttagcgattgtaattgcaccgcgtttgcaaatgcggatatccggaatggtgggacaggttgtgtgatttggaccggacagcttgacgatatgcggaattacgctattggcgctaccgacggtcaagatctttatgtcagattggctgctgctgatattgctaagaagagaaacgcgaacggggaaatcataagttt

SEQ ID NO: 69 - DNA, 1316 bp, Brassica rapa
tacaccttctcatccttgctcgtcttcattgtcttgattctgtttcatcccgccctttcgatctatatcaacattttgtcgtctacagaaactcttaccatctcaggcaacagaacacttgtatctcccggtgatatcttcgagctcggtttcttcaagaccacttcacgttctcgttggtatctcgggatatggtacaagaaaatctccgaaagaacctatgtatgggttgccaacagagacaaccctctatccattgccgttggaaccctcaaaatctcaggcaataatcttgtcctccttggtcaatccaataaatctgtttggtcgacaaatctaactagagaaaatgagagatctccgatggtggcagagcttctcgctaacggaaacttcgttttgcgagactccgataacaacggtgcaagtggattcttgtggcaaagtttcgattaccctacagatactttgcttccagagatgaaactcggttacgaccacaaaacagggcagaacaggttccttttgtcatggagaagttcagatgatccttcaagcggggattacttgtacaagctcgaaacccgaaggtttcctgaattttatctatcgagtggggtctttcgattgcataggagtggtccatggaatggaatccgatttagtggcatactagatgaccaaaagttgagttacttggcgtacaatttcacagagaatagtgaagaagtcgcttatacattccgaatgatcaacaacagcatctactcgagattgacagtaagtttctcagggtattttgaacgacagacatggaatccgtcattagggatgtggaacatgttctggtcttttccactggactcacagtgcgatggctacaggatgtgtgggccttacgcttactgtgacgcgaacacatcaccgatttgtaactgtatacaagggttcaatcccttagatgcggagcagtgggatctgagaagctggtcaggtgggtgtataaggaggacgcagcttagctgcaatggagatggttttacaaggatgaggaatatgaagttgccagaaactacgatggctattgtcgaccgcagtattggtgagaaagaatgccagaagaggtgccttagcgattgtaattgtaccgcgtttgcaaatgcggatatccggaatggtgggacgggttgtgtcatttgggcaggagagcttatcgacatgcggaattacggtgctgacggtcaagatctttatgtcagattagctgcagctgacctcgttgagaagagaaacgcgaattggaaaatcataagttt

SEQ ID NO: 70 - DNA, 1316 bp, Brassica rapa
accttttccttcttgatcgtcctcgctgtcttgattctttttcatcctgccctttcgatttatgtaaacactttgttgtctacagaatctcttacaatctcaagcaacagaacacttgtatctcccggtaatgtcttcgagcttggattcttcactcccggatcaagttctcgctggtatctcgggatatggtacaagaaattatccgacagaacctatgtatgggttgccaacagagatagccctctctccagttccattgggaccctcaaaatctccaacatgaaccttgtcctccttgatcactccaataaacctgtttggtcgacgaatcttacaagaggaaatgagagatcaccggtggtggcagagcttctcgctaacggaaacttcgtgatgcgatactccaataacaacgactcaagtggattcttgtggcaaagtttccattaccctacagatactttgcttccagagatgaaactgggttacgaccgcaaaacaaggctgaacagataccttacatcatggagaaattcagatgatccgtcaagtggggaaatctcgtacttcctagacattcaaacgggaatgcctgagttctatctattgcaaagcggcgcacgaatgcaccgaagcggtccatggaatggagtccgatttagtggcatgccaggggaccaaaagttgaattacatggtgtacaatttcacagagaatagtgaggacgttgcttatacatttcgaatgaccaacaagagcatctactcgagattgaaagtaagttccgaagggtttttggagcgactgacgtggaccccaaattcaattacatggaacatgttctggtatttaccattggaaaaccagtgcgatatttacatgatttgtgggcgttacgcttactgtgacgtgaacacatcaccgttgtgtaactgtatccaagggttcaatcgctcgaatgaggagcggtgggatctgaaagattggtcaagcgggtgtataaggaggacgcggctgagttgcagtggtgatggttttacaaggatgaggaagatgaaattgccagagactaagatggcgattgtcgaccgcagtattggtgtgaaagaatgtgaaaagaggtgccttagcgattgtaattgtacagcgtttgcaaatgcggatatccggaatggtgggacgggttgtgtgatttggactggagacctcgaggatcttcgaaattactatgctgacggtcaagatctttatgtcagagtggctgccgctgatcttgttaaaaagagcaacgcgaattggaaaatcataagttt

SEQ ID NO: 71 - DNA, 1316 bp, Brassica rapa
acctccatcttgctcgtctttgttgtcatgattctatttcatcctgccttttcgatctatatcaactctttgtcgtctgcaggctctcttacaatctcaagcaacagaacacttgtatctcccggtaatatcttcgagctcggcttcttcagaaccaactcaagttctcgttggtatctcgggatatggtacaagcaattgtccgagagaacctatgtatgggttgccaacagagataaccctctctccaattccattggaaccctcaaaatttccgatatgaacctcctcctcgttgatcactctaataaatctgtttggtcgacgaatcttaccagaggaaatgagagatcatctctggtggtggcagagcttctcgctaatggaaacttcgtgatgcgagactccaataacaacgacgcaggtggattcctgtggcaaagtttcgattaccctacagatactctgcttccagagatgaaactgggttacaacctcaaaaagggactgaacaggttccttacgtcgtggagaagttcagaggatccctcaagcggggaaatctcgtacaagctcgaaatgagaaggcttcctgagttttatctatggaatgaggactttccaatgcatcggagtggtccatggaatggaatcgaatttagtggaataccagaggaccaaaagtcgagttacatggcgtacaatttcacagagaatagtgaaggagtcgcttatacattccgaatgaccaacaacagcatctactcgagattgacagtaagttcagaagggaattttgagcgactgacgtggaatccgttattggggatgtggaacgtgttctggtcttctccagtggacgcccagtgcgatatgtacaggacgtgtgggccttactcttactgtgacgtgaatacatcgccggtttgtaactgtatccaagggttcaatccctcgaatgtgcagctgtgggatctgagagacggggcaggtgggtgcataaggaggacgcggctgagctgcagtggagatggttttaccaggatgaagaatatgaagttgccagaaactacgatggctactgtcgaccgcagttttggtctgaaagaatgtgagaagaggtgccttagcgactgtaattgtaccgcgtttgcaaatgcggatatccggaatggtgggacaggttgtgtgttttggaccggacggcttgacgatatgcggaattacgctgctgatcacggtcaagatctttatgtcaaagtggctgcggctgatctcgttaagaagagaaacgcgaatgggaaaaacataagttt

SEQ ID NO: 72 - DNA, 1304 bp, Brassica rapa
accttctcctttttgctagtcttcgttgtcatgattctgtttcatcccgccctttcgatctatttcaacactttgtcgtctacagaatctcttacaatctcaaccaacagaacacttgtatctcccggtgatgtcttcgagctcggcttcttcagaaccaattcacgttggtatctcgggatgtggtacaagaaattgccctatagaacctatgtatgggttgccaacagagataaccctctctccagttccattggaacgctcaaaatctcaggcaataatcttgtcctccttggtcattccaataaatcagtttggtcgacgaatcttactagaggaaatgagagatccccggtggtggcagagcttcttgctaacggaaacttcgtgatgcgagactccaatatcaacgacgcaagtggattcttgtggcaaagttttgattttcctacaaatactttgcttccagagatgaaactgggtttcaaactcaaaacagggctgaacaggttccttacatcatggagaagttcaaatgatccgtcaagcggcaatttctcgtacaagctcgaagcccaaaggcttcctgagttttatttatggaatgagaaatttccatggcatcggagtggtccatggaatggaatcgaatttagtggcataccagaagacaaagagttgagttacatggtgtacaatttcacagagaatagtgaagaggtcgcttatacattcctaatgaccaacaacagcatctactctagattgacaataaattccgcagggtattttcagcgactgacgtgggatccgttattaggaatgtggaacgtgttctggtcttctccagtggacctccagtgtgattcgtacaggaggtgtgggccttatgcttactgtgacgtgaccacatcaccggtttgtaactgtatccaaggattcaatcccaggtttgtggagcggtgggatatcagagactggtcagctgggtgtataaggaggacgcgtcttagctgcagtggagatggttttacaaggatgaagaatatgaagttgccagaaactacgatggctattgtcgaccgcactattggtctgaaagaatgtagaaagaggtgcgttcgcgattgtaattgtaccgcgtttgcaaatgcggatatccggaatggtgggacgggttgtgtgatttggaccgtactgcttgaggatatgcggaattacgctgacggtcaagatctttatgtcagattggctgctgctgatctcgtcaagaagagaaatgcgaatgggaaaatcataagttt

SEQ ID NO: 73 - DNA, 1292 bp, Brassica rapa
accttctcctacttgctcgtcttcgttgtcatgattctatttcatcctgccctttcgatctatatcaacactttgtcgtctacagaatctcttacaatctcaagtaacagaacacttgtatcacccggtagtatcttcgagctcggcttcttcagaaccaattctcgttggtatctcgggatgtggtacaaggaattgtccgagagaacctatgtatgggttgccaacagagataaccctatctccaattccattggaactctcaaaatctcaggcaataatcttgtcctccttggtcactccaataaatctgtttggtcgacgaatcttactagagaaaatgagagatctccggtggtggcagagcttcttgctaatggaaacttcgtgatgcgagactcaagtggatttttgtggcaaagtttcgatttccctacagatactttgcttccagagatgaaactgggttacgacctcaaaacaaggttgaataggttccttgtatcatggagaagtttaaatgatccttcaagcgggaatttctcgtacagactcgaaacccgaaggcttcctgagttttatctatcaaaacgcgacgttccagtgcatcggagcggtccatggaatggaatccgatttagtggcataccagaggacgaaaagttgagttacatggtgtacaatttcacagagaatagtgaggaggctgcttatacattcctaatgaccaacaacaacatctactcgagattgacaataagttcggatgggagttttcagcgactgacgtggactccgtcatcaggcgcgtggaacgtgttctggtcttctccagtaaaccccgagtgcgatttgtacatgatttgtgggcctgacgcttactgtgacgtgaacacatcaccgtcgtgtatctgtatccaagggttcaatcccaaggatctgccgcagtgggatctgagagactggacaagtgggtgcataaggaggacacggctgagctgcaggggcgatggttttacaaggatgaagaatatgaagttgccagaaactacgatggctattgtcgaccgcagtattggtatcaaagaatgtaagaagagatgccttagcgattgtaattgtacagcttttgcaaatgcggatatccggaatggtgggacgggttgtgtgatttggaccggacagcttgacgatatccggaattacggtactgacggtcaagatctttatgtcagattggctgcagctgatctcgttaagaggagaaacgcgaatgggaaaatcataagttt

SEQ ID NO: 74 - DNA, 1334 bp, Brassica rapa
accttctccttcttgctcgtcttcgttgtcttgattctatttcatcctgccctctcgatctatttcaacattttgtcgtctacagaaactctcaccatctctggcaacaaaacacttgtatctcccggtgatgtcttcgagcttggttttttcactccccgatcaagttctcgctggtatctggggatatggtacaagaaactctacttcagaatcaaaacctacgtatgggttgccaacagagatagccctctcttcaatgccattggaaccctcaaaatctcaggcaataatcttgtcctccttgatcactctaataaatcggtttggtcgacgaatcttactagaggaaatgagagatctctggtggtggcagagcttctcgccaatggaaacttcgtgatgcgagactccgatatcaacgacgcaactggattcttgtggcaaagtttcgattaccctacagatactttgcttccagagatgaaactgggttacgaccgcaaaaaagggctgaacagattccttacatcatggagaaattcagatgatccctcaagcggggaaacctcgtacaaactagacactcaaaggggtttgcctgagttctatctattgataaacggctcacgaggccaacggagcggtccatggaatggaatccgatttagtggcataccagaggacctaaggttgagttacatggtgtacaatttcatagagaatagtgaggaggtcgcttatacatttcgaatgaccaacaacagcatctactcgagattgaccataagttccgaagggcttttggagcgatggacgtgggccccggcatcattttcatggaacttgttctggtctttaccagcggatacctggtgcgatgtttacatggcttgtgggccgtacgcttactgtgacgtgaacacctcaccggagtgtaactgtatacaagggttcaaccgctccaatgagcagcagtgggatctgcgagacgggtcagctgggtgtgtaagggggacgcggctgagctgcaatggtgatggttttaccaggatgaagaggatgaagttgccagaaactacgatggcgattgtcgaccgcagtattggtattggtgtgacagaatgtgagaagagatgccttagcgattgtaattgtaccgcgtttgcaaatgcggatatccggaatggtgggacgggttgtgtgatttggaccacagggcttgaggatatccggacttactttgctgatgatattggtcaagatctttatgtcagattggctgccgctgatcttgttaaaaagagaaacgcgaatgggaaaatcataccttt

SEQ ID NO: 75 - DNA, 1322 bp, Brassica rapa
attttctccttcttgctcgtattcgttctcttgattctatttcatcctgtcctttcgatctatatcaacactttgtcatctacagaatctcttaccatatcaggcaacagaacacttgcatctccgggtgatgacttcgagctcggtttcttcaaaaccatttcacgttctcgttggtatctcgggatatggtacaagaaaatctcccaaagaacctacgtatgggttgccaacagagatagccctctcttcaatgccgttggaaccctcaaaatctcaggcaataatcttgtcatccttggtgactccaataattctgtttggtcgacgaatcatactagaggaaatgagagatctccggtggtggcagagcttctcgctaacggaaacttcgtcatacggtactccaataacaacgacgcaagtggattcttgtggcaaagtttcgattaccctacagatactttgcttccagagatgaaactggggtacgatctcaaaaaggggatgaacagattccttacatcatggagaaattcagatgatccctcaagcgggaatatcaagtaccaactagacactcaaaggggaatgcctgagttctatctattgaaagagggctctcgagcccaccggagtggtccatggaatggtgtccaattttatggcataccagaggaccaaaagttgagttacatggcgtataatttcatagagaatagtgaggaggttgcttatacatttcgaatgaccaacaacagcatctactcgagattgaaaataaattccgatgaatatttggatcgattgacgtggaccccgacatcaactgcatggaacttgttctggtctgcaccagtggatatccggtgcgatgtatacatggcttgtgggcctgacgcttactgtgacgtgagcacatcaccggtttgtaactgtatccaaggattcaagcgcagcgatgagcagcagtgggatctgagagacccgtcaagtgggtgtataagggggacgccgctgagctgcaagggagatggttttacaaggatgaagaagatgaagttgccagaaactaggatggctattgtcgaccgcagtattggtgtaaaagaatgtgaaaagaggtgccttagcgattgtaactgtactgcgtttgcaaatgcggatatccggaatggtgggacgggttgtgtgatttggaccagagagcttgaggatatccggacttactctgctgctgatcttggccaagatctttatgtcagattggctgccgctgatcttgttaaaacgagaaatgagaatggaaaaatcataacttt

SEQ ID NO: 76 - DNA, 1313 bp, Brassica rapa
accttctccgtcttgctcgtcttcgttgtcatgtttctatttcatcctgccctttcgatctatatcaacactttgtcgtctaccgaatctcttacaatatcaagcaacagaacacttgtatctcccggtgatgtcttcgagttcggtttcttcaaaaccaactcaagctctcgttggtatctcgggttatggtacaagaaattgccctatagaacctatgtatggattgccaacagagataaccctctctccaattccattggaaccctcaaaatctccgacatgaacctcgtcctccttgatcactctaataaatctgtttggtcgacgaatcttactagaggaaatgagagatctccggtggtggcagagcttctccctaacggaaacttcgtgatacgatacttcaataataacgacgcaagtggattcttgtggcaaagtttcgattaccctacagatactttgcttccagagatgaaactgggttacgacctcaaaaagggactgaacagattccttacatcatggagaagttcagatgatccatcaagcggggaattctcgtacaagctcgaaccccgaaggcttcctgagttttatatatttattgaagacattccagtacatcggagtggtccatggaatggaatccgatttagtggcatactagaggaccaaaagttgagttacatggtgtacaatttcacagagaatagtgaagaggtcgcttatgcattccgaatgaccaacaacagcatctactcgagattgacattaagttccgaagggtattttcagcgactgacgtggactccatcgtcagtggtatggaacctgttctggtcttctccagcgaacgtcgagtgcgatttgtacagggtttgtgggcctaacggttactgtgacatgaacacatcaccgtcgtgtaactgtatccaagggttcaatccccgtaatatgcagcagtgggatctgagagatccgtcaagtgggtgtataaggaggacgttgctgagctgcggtggggatggttttaccaggatgatgaaggtgaagttgccagacactacgatggctattgttgacaggagtattggtctgaaagaatgtaagaaaaggtgccttggcgattgtaattgtaccgcgtttgcaaatgcagataccaggaatggtgggacgggatgtgtgacttggaccggtgagcttgaggatatccggaattacattagggacggtcaagatctttatgtcagattggctgcagctgatctcgttaagaagagaaacgcgaacggtaaaatcgtaagttt

SEQ ID NO: 77 - DNA, 1292 bp, Brassica rapa
accttatccgtcttgctcgtcttcgttgtcatgattctgtttcatcctgccctttcgatctatatcaacactttgtcgtctacagaatctcttacaatctcaagcaacagaacacttgtatctcccggtaatatcttcgagctcggcttcttcagaaccaattctcgttggtatctcggggtgtggtacaaggaattgaccgagataacctatgtatggattgccaacagagataaccctatctccaattccattggaatcctcaaaatctcaggcaataatcttgtcctccttggtcactccaataaatctgtttggtcgacgaatcttactagagaaaatgagagatctccggtggtggcagagcttctcgctaatggaaacttcgtgatgcgagactcaagtggattattgtggcaaagtttcgattaccctacagatactttacttccagagatgaaactgggttacgacctcaaaacagggttgaacaggttccttatatcatggagaagtttagatgatccatcaaacgggaatttctcgtacagccttgaaaaacgagagcttcctgagttttatctatataaaggcaactttcgagtgcatcggagtggtccatggaatggaatcgcatttagtggcataccagaagaccaaaagttgagttacatggtgtacaatttcatagagaatagtgatgaggccgcttatacattccgaatgaccaacagcagcatctactcgaaattgacaataaattcggaagggagatttcagcgactgacgtggactccatcatcaggcgcgtggaacgtgttctggtcttctccagagaaccccgagtgcgatttgtacatgatttgtgggccttacgcttactgtgacttgaacacatcaccgtcgtgtaactgtatccaagggttcaatcccggggatgtggagcagtgggatctgagagactggacaagtgggtgcataaggaggacacggctgagctgcagtgacgatggttttacaaggatgaagaatatgaagttgccagaaacaacgatggctattgtcgaccgcagtattggtatgaaagaatgtaagaagagatgccttagcgattgtaattgtacagcttttgcaaatgcggatgtccggaatggtgggacgggttgtgtgatttggaccgcacagcttgacgatgtccggaattacggtgctgacggtcaagatctttatgtcagattggctgctgctgatctcgttaagaggagaaacgcgaatgggaaaatcataagttt

SEQ ID NO: 78 - DNA, 1301 bp, Brassica rapa
atcttctccttcgttgtcatgattctatttcatcctgcactttcgatctatattaacactttgtcggctacagaatctcttacaatctcaagcaaaagaacacttgtatctcccggtaatgtcttcgagctcggtttcttcaagaccaccttaagttctcgttggtatctcgggatatggtacaagaaagtgtccgagagaacctatgtatgggttgccaacagagataaccctctctccaactccattggaaccctcaaaatctcaggcaataatcttgtcctccttggtcactccaataaatctgtttggtcaacgaatctaactagaggaaatgagagatctccggtggtggcagagcttctcgcaaatggaaacttcgtgatgcgagactccaataacaacgacgcaagtggattcttgtggcaaagtttcgattaccctacagatactttgcttccagagatgaaacttggttacgacctcaaaacagggctgaacagatttcttatatcatggagaagttcaaatgatccatcaagcggaaatttctcgtacaagctcgaaaaccgagagcttcctgagttttatctacagcaaaacgacattcgagcgcatcggagcggtccatggaatggtatcggatttagcgccataccagaggaccgaaagttgagttacatggtgtacaatttcacagagaatagtgaagaggtcgcttatacatttctaatgaccaacgacagcatttactcgagaattcaaatgagttccgaaggggatttgcggcgactgatgtggacgccgacatcatgggaatggagtttgttctggtctgcaccagtggatcctcagtgcgatgtgtacaagacatgtggaccttacgcttactgtgacctgaacacatcaccattgtgtaactgtatccaagggttcatgccctcgaatgtgcagcagtgggatctgagaaacccgtcaagtgggtgtataagaaggacgcggctgggctgcagtggtgatggttttaacaagatgaagaatatgaagttgccagagactacgacggctattgtcgacaggagtattggtatgaaagaatgtaagaagagatgccttagcgattgtaattgcacagcgtttgcaaatgcggatatccggaatggtgggactggttgtgtgatttggaccgaacggcttcatgatatcaggaattactttgataacggtcaagatctttatgtcagattggctgccgctgatctcggtcaagagagaaacgcgaatgggaaaatcataatttt

SEQ ID NO: 79 - DNA, 1313 bp, Brassica rapa
accttctcctttttgctagtcttcgttgtcatgattctgtttcatcccgccctttcgatctatatcaacactttgtcgtctacagaatctcttaaaatctcaagcaacagaacacttgtatctcccggtagtatcttcgagctcggcttcttcagaaccaattctcgttggtatctcgggatatggtacaagaaacttccctatagaacttatgtatgggttgccaacagagataaccctctttccaactccactggaaccctcaaaatctcaggcaataatcttgtcatccttggtcactccaataaatctgtttggtcggcgaatcttacaagaggaagtgagagatctacggtggtggcagagcttctcgctaacggaaacttcgtgatgcgagactccaataaaaacgacgcaatcttgtggcaaagtttcgatttccctacagatactttgcttccagagatgaagctgggttacgacctcaaaacagggctgaacaggttccttacatcatggagaagctcagatgatccctcaagcgggaatttctcgtacaagctcgaaaaccaaaggcttcctgagttttatctatcgagtcatggaatttttcgattgcatcgaagtggtccatggaatggaatcggatttagtggcataccagaggacgaaaagttgagttacatggtgtacaatttcacagagaatagtgaagaggttgcttatacattccgaatgacaaacaacagcatctactcgagattgacactaagttccaaaggggattttcagcgactgacttgggatccgtcattagaaatatggaacatgttctggtcttctccagtggacccccagtgcgattcatacataatgtgtggggcgtacgcttactgtgacgtgaacacatcaccggtatgtaactgtatccaagggttcaatccccggaatatacagcggtgggatcagagagtctgggcaggtgggtgtgtaaggaggacgcagctgagctgcaatggagatggttttactaggatgaagaatatgaagttgccagaaactacgatggctattgtcgaccgcagtgttggtgtgaaagaatgtgagaagaggtgccttagcgattgtaattgcaccgcgtttgcaaatgcggatatccgtaatggtgggacgggctgtgtgatttggaccggacagcttgacgatatgcggaattacgctattggcgctaccgacggtcaagatctttatgtcagattggctactgctgatattgctgagaagagaaacgcgaacggggaaatcataagttt

SEQ ID NO: 80 - DNA, 1377 bp, Brassica rapa
atgcaaggtgtacgatacatctatcatcattcttacaccttcttgctcgtcttcgttgtcatgattctatttcatcctgccctttcgatctatatcaacactttgtcgtctacagaatctcttacaatttcaaacaatagaacacttgtatctcccggtgatgttttcgagctcggtttcttcaaaaccacctcaagttctcgttggtatctcgggatatggtacaagcaattgcccgagaaaacctatgtatgggttgccaacagggataaccctctccccaattccattggaaccctcaaaatatccaacatgaaccttgtcctccttgatcactctaataaatctgtttggtccacgaatcttactagacgtaatgagagaactccggtgatggcagagcttctcgctaatggaaacttcgtgatgagagactccaataacaacgatgcaagtgaattcttgtggcaaagtttcgattaccctacagatactttgcttccagagatgaaactgggttacgacctcaaaacagggttgaacaggttccttatatcatggagaagttcggacgatccgtcaagcggggattactcgtacaagctcgaaccccgaaggcttcctgagttttatctactgcaaggagacgttcgagagcatcggagtggtccatggaacggaatccgatttagtgggatactagaggaccaaaagctgagttacatggagtacaatttcacagagactagtgaggaggtcgcttatacattccgaatgaccaacaacagcttctactcgagattgacactaagctccacagggtattttgagcgactgacgtgggctccgtcatcagtgatatggaacgtcttctggtcttctccagcaaacccccagtgcgatatgtacaggatgtgtgggccttactcttactgtgacgtgaacacatcaccatcgtgtaactgtatacaagggttcgatcccaggaatttgcagcagtgggctctgagaatctcattaagggggtgtaaaaggaggacgctgctgagctgcaatggagatggttttaccaggatgaagaatatgaagttgccagaaactacgatggccattgtcgaccgcagtataggtgagaaagaatgtaagaagaggtgccttaccgattgtaattgtaccgcgtttgcaaatgcggatatccggaatggtgggacgggttgtgtgatttggactggaaatctcgctgatatgcggaattacgttgctgacggtcaagacctttatgtcagattggctgcggctgatctcgttaagaagagtaacgcgaatgggaaaatcataagtttgactgttggagttactgttctgcttcttctgatc

SEQ ID NO: 81 - DNA, 1316 bp, Brassica rapa
accttctccttcttgctcgtctttgttgtcttgaatctatttcatcctgccctttcgatctatttcaacattttgtcatctacagaaactcttaccatctcaggcaacagaacacttgtatctccaggtaatgtcttcgagctcggtttcttcaaaaccacctcaaattctcgttggtatcttgggatatggtacaagaaactctacttccgaacctacgtatgggttgccaacagagatagccctctctccactggaaccctcaaaatctcagggaataatcttgtcctccttggtcactccaataaatctgtttggtcgacgaatcttacaagaagaaatgagagatctccggtgatggcagagcttctcgctaatggaaacttcgtgatgcgagactccaataacaacgacgcaagtggattcctgtggcaaagtttcgatttccctacagatactttgcttccagagatgaaactgggttacgaccacaaaaaaaggctgaacagattccttacatcatggagaaattcagatgatccgtcaagcggggaattctcgtaccaactagacactcaaaggggaatgcctgagttcttagtattaaaagaaggctatccaggccaccggagcggtccatggaatggagtccgatttagtggcattccagaggaccaaaagttaagttacatggtgtacaatttcacagagaatagtgaggaggttgcttattcatttcgagtgaccaacaacagcatctactcgagactgaaaatcaattccgaagggtttttggagcgattgacgtggaccccagcatcaagtgcgtggaacttgttctggtctgtaccagtggatacccggtgcgatgtgtacatgtcttgtgggccttacgcttactgtgacgtgaacacatcaccggtttgtaactgtatccaagggttcaatcgctccaatgagcagcagtgggatatgagagacggggcaagtgggtgtataagggggacgcagctaagctgcagtgatgatggttttacaaggatgaagaagatgaaattgccagacactacgatggctattgtcgaccggagtattggtgtgaaagaatgtgagaagaggtgccttagcgattgtaattgtacggcgtttgcaaatgctgatatccggaatggtgggacgggttgtgtgatttggactggagagcttgaggatatccggaattactttgctgttcttggtcaagatctttatgtcagattggctgccgctgatcttgctaaaaagagaaatgcgaatggaaaaatcgtaactttgac

SEQ ID NO: 82 - DNA, 1307 bp, Brassica rapa
accttatactttttgcttgtctttttcgtcttgattctaccacgtcctgccttttcaatcaacactttgtcgtctacagaatctcttacaatctcaagcaacagaacacttgtatctcccggtaatttcttcgagctcggcttcttcagaaccaattctcgttggtatctcgggatgtggtacaagaaattgtccgtcagaacctacgtatgggttgccaacagagataaccctgtagccaactccgttggaaccctcaaaatctcaggcaataatcttgtcctccttggtcactccagtaaatctgtttggtcgacgaatcttactagaagaaatgagagatcgtctgtggtggcagagcttctcgctaacggaaactttgtgatgcgagactccaataacaacgacgcaagtagattcttgtggcaaagtttcgattacccaacagatactttgcttccagagatgaaactgggttacgacctcaaaacagggctgaataggttccttacagcatggagaagttcagatgatccatcaagcggggaaatctcgtacaagctcgaaccccgaagacttcctgagttttatctattgaaacgccgcgtctttcgactgcatcggagtggtccatggaatggaatccgatttagtggcataccagaggaccaaaagttgagttacatgatttacaatttcacagagaatagtgaagagctcgcttatacattccgaataaccaacaacagcatctactcgatattgacagtaagctccgaagggaaattagagcggctgatgtggaatccgtcattagcgatgtggaacgtgttctggttttttccagtggactctcagtgcgatacgtacatgatgtgtgggccttactcttactgtgacgtgaacacatcaccggtatgtaattgtatccaagggttcaatcccatgtacgtggaggagtgggatctgagagagtggtcaagtgggtgtataaggaggacgctgcttagctgcagtgaagatggttttaccaggatgaagaatatgaagctgcctgacactacgatggcgattgtcgaccgcagtattggtctgaaagaatgtgagaagaggtgccttagcgattgtaattgtaccgcgtttgcaaatgcggatatccggaatggtgggacgggttgtgtgatttggaccgggaaggttgaggatatgcggaattatggtgctgacggtcaagatctttatgtcagattggctgcagctgatattatcgataagaagagaaacgccaatgggaaaatcataagtct

SEQ ID NO: 83 - DNA, 1316 bp, Brassica rapa
accttctccttcttgctcgtcttcgttgtctcgattatatttcatcctgccctttcgatctatatcaacactttgtcatctacagaatctcttaccatatcaggcaacaggacacttgtatctcccggtgatgacttcgagctcggtttcttcaaaaccacttcacgttctcgttggtatctcgggatatggtacaagaaaatctccgaaagaacctacgtatgggttgccaacagagatagccctctctccaatgcagttggaaccctcaaaatctctggcaataatcttgtcctcctcgatcactttaataaatctgtttggtcgacgaatcttactagaggaaatgagagatctccggtggtggcagagcttctcgccaatggaaacttcgtgatacgatacttcagtaacaacgacgcaagtggattcctgtggcaaagtttcgattaccctacagatactttgcttcccgagatgaaactgggttacgacatcaaaaccgggctgaacagattccttacatcatggagatcctacgatgatccctcaagcggggaaatcgtgtacaaactagacactcaaaggggaatgcctgagttttttctattaaaaaatgactttccagcgcatcggagtggtccatggaatggtatcggatttagtggcctacctgaggaccataagttgggttacatggcgtacaatttcatagagaatagtgaagaggttgcttattcatttcgaatgaccaacaacagcatttactcgagattggaaataaattccgacggagatttggagcgactgatctggactccgacatcatgggaatggagcttgttctggtcttcaccagtggatctccagtgcgatgtgtacaagacttgtgggccttacgcttactgtgacttgaacacatcaccattgtgtaactgtatccaagggttcacgccctcgaatgtgcagcagtgggatctgagaaacccgtcagctgggtgtataaggaggacgaggctgagctgccgtggtgatggttttacaaggatgaagaatatgaagttaccagaaactacgatagctacggtcgaccgcaatattggtctgaaagaatgtaagaaaatgtgccttagcgattgtaattgtaccgcgtttgcaaatgcggatatccggaatggtgggacgggttgtgtgatttggactggacggcttcatgatatccggaattacgctgctgacggtcaagatctttatgtcagattggctgccgttgatctcgctcaaaagagaaacgcgaatgggaaaatcataacttt

SEQ ID NO: 84 - DNA, 483 bp, Artificial Sequence
  probe specific to S haplotype of Brassicaceae
tcagatgatccgtcaaccggggatttctcgtacaagctcgaactccgaaatattcctgagttttatctattgcaaggcgactttccagagcatcggagtggtccatggaatggaatccaatttagtggcataccagaggaccaaaagttgagttacatggtgtacaatttcacagagaatagtgaggaggtcgcttatacgttcctaatgaccaacaacagcttctactcgagattgataataagttccgaagggtattttcggcgactgacgtgggctccgtcatcagtgatatggaacgtgttctggtcttctcctaatcaccagtgcgatatgtacaggatgtgtgggccttactcttactgtgacgtgaacacacaaccgatttgtaactgtatccaagggttcaatcccgggaatgtgcagcagtgggctttgagaatcccaataagtgggtgtaaaaggaggacgccgctgagctgc

SEQ ID NO: 85 - DNA, 486 bp, Artificial Sequence
  probe specific to S haplotype of Brassicaceae
tcagatgatccgtcaagtggggatttctcgtacaagctcgaaggttcgagaaggcttcctgagttttatctaatgcaaggcgacgttcgagagcatcggagtggtccatggaatggaatccaatttagtgggataccagaggaccaaaagttaagttacatgatgtacaatttcacagataatagtgaggaggtcgcttatacattcctaatgaccaacaacagcttctactcgagattaaaattaagttccgaagggtatttggagcgactgacgtgggctccgtcatcagggatatggaacgtgttctggtcttctccgaaccaccagtgcgatatgtacaggatgtgtggaacttactcttactgtgacgtgaacacatcaccgtcatgtaactgtatcccagggttcaatcccaagaaccggcagcagtgggatctgagaatcccaataagtgggtgtataagaaggacgcggcttggctgc

SEQ ID NO: 86 - DNA, 486 bp, Artificial Sequence
  probe specific to S haplotype of Brassicaceae
tcagaggatccctcaagcggggaaatctcgtacaagctcgaaatgagaaggcttcctgagttttatttatggaatgaggactttccaatgcatcggagtggtccatggaatggaatcgaatttattggaataccagaggaccaaaagtcgagctacatggcgtacaatttcacagagaatagtgaaggagtcgcttatacattccgaatgaccaacaacagcttgtactcgagattgacagtaagttcagaagggaattttgagcgactgacgtggaatccgttattagggatgtggaacgtgttctggtcttctccagtggacgcccagtgcgatatgtacaggacgtgtgggccttactcttactgtgacgtgaatacatcgccggtttgtaactgtatccaagggttcaatccctcgaatgtgcagctgtgggatctgagagacggggcaggtgggtgcataaggaggacgcggcttagctgc

SEQ ID NO: 87 - DNA, 489 bp, Artificial Sequence
  probe specific to S haplotype of Brassicaceae
tcggatgatccttcaagcggggaaatctcgtaccaattagacactcaaaggggaatgcctgagttctatctattgataaacggctcacgataccaccggagcggtccatggaatggagtccaatttaatggcataccagaggaccaaaagttgagttatatggtgtacaattacatagagaatgatgaggaggttgcttattcatttcgaatgaccaacaacagcatctactcgagattgacaataagtttcgaagggtttttggagcgatatacgtggaccccgacatcgattgcatggaacttgttctggtcttcaccagtggatatccggtgcgatgtgtacatggcttgtgggcctgacgcttactgtaacttgaacacatcaccattgtgtaactgtatccaagggttcaagcgctccaatgagcagcagtgggatgtgagagacgggtcaagtgggtgtataagggagacgcggctgagctgc

SEQ ID NO: 88 - DNA, 486 bp, Artificial Sequence
  probe specific to S haplotype of Brassicaceae
tcagatgatccgtcaagcggggattactcgtacaagctcgaaccccgaaggcttcctgagttttatctactgcaaggagacgtccgagagcatcggagtggtccatggaacggaatccgatttagtgggatactagaggaccaaaagttgagttacatggtgtacaatttcacagagaatagtgaggaggtcgcttatacattccgaatgaccaacaacagtttctactcgagattgacactaagctccacagggtattttgagcgactgacgtgggctccgtcatcagtgatatggaacgtcttctggtcttctccagcaaacccccagtgcgatatgtacaggatgtgtgggccttactcttactgtgacgtgaacacatcaccatcgtgtaactgtatacaagggttcgatcccaggaatttgcagcagtgggctctgagaatctcattaagggggtgtaaaaggaggacgctgctgagctgc

SEQ ID NO: 89 - DNA, 486 bp, Artificial Sequence
  probe specific to S haplotype of Brassicaceae
tgggatgatccatcaagcggggatttcttgtacgagctcgaaacccgaaggcttcctgaattttatctaacgattgggatctttcgagtgcatcggagtggtccatggaatggaatccgatttagtggcataccagatgaccaaaagttgagttacttggtgtacaattttacagagaatagtgaagaagttacttatacattccgaatgaccaacaacagcatctactcgagattgatagtaagtttctcagggtatattcagcgacagacgtggaatccgacattagggatgtggagcgtattctggtcttttccattcgactcacagtgcgattcgtacagagcgtgtgggcctaacgcttactgtgacgtgaatacatcaccgttttgtaactgtatccaagggttcattccctcgaatgtggtgcagtgggatcagagagtctgggcaggtgggtgtataaggaggacgcgtcttagctgc

SEQ ID NO: 90 - DNA, 486 bp, Artificial Sequence
  probe specific to S haplotype of Brassicaceae
tcagatgatccgtcaagcggggatttcttgtacgagctcgaagcccggaggcttcctgaattttatctatctagtgggatctttcgattgtatcggagcggtccatggaatggaatccgatttagtggcataccagatgaccaaaagttgagttacatggtgtacaatttcacagagaatagtgaagaagtcgcttatacattccgaatgaccaataacagcatttactcgaaattgacagtaagtgtctcagggaagtttgagcgacagacgtggaatccgacattagggatgtggaacgtgttctggtctttcccgtcggactcacagtgcgatacgtacaggatttgtgggccttattcttattgtgacgtgagcacatcaccgatttgtaactgtatccaagggttcaatccctcgaatgtgcagcagtgggatcagagatcctggtcaggtgggtgtataaggaggacgcaacttagctgc

SEQ ID NO: 91 - DNA, 486 bp, Artificial Sequence
  probe specific to S haplotype of Brassicaceae
ttagatgatccgtcaagcgggaatttctcgtacagcctcgaaaaacgagagcttcctgagttttatctatataaaggcgactttcgagtgcatcggagtggtccatggaatggaatcgcatttagtggcataccagaggaccaacagttgagttacatggtgtacaatttcacagagaatagggatgaggccgcttatacattccgaatgacgaacagcagcatctactcgaaattgacaataaattcggaagggagatttcagcgactgacgtggactccatcatcaggcgcgtggaacgtgttctggtcttctcctgtgaaccccgagtgcgatttgtacatgatttgtgggccttacgcttactgtgacttgaacacatcaccgtcgtgtaactgtatccaagggttcaatcccggggatgtgcagcagtgggatctgagagactggacaagtgggtgcataaggaggacacgcctgagatgc

SEQ ID NO: 92 - DNA, 489 bp, Artificial Sequence
  probe specific to S haplotype of Brassicaceae
tcagatgatccatcaagcggggaaatctcgtacaaactagacactcaaacgggaatgcctgagttctatctattgcaaacgggcgtacaagtgcatcggagcggtccatggaatggagtccgatttagtggcataccaggggaccaagagttgagttacatggtgtacaatttcacagagaatactgaggaggttgcttatacatttcgaatgactgacaacagcatctactcgagattgaaagtaagttccgaagggtttttggagcgactgacgtggaccccgaactcaactacatggaacttgttctggtatttaccattggaaaaccagtgcgatatgtacatgatttgtgggcgttacgcttactgtgacgtgaacacatcacccttgtgtaactgtatccaagggttcataccctggaataagcagcagtgggatcagagagacccgtcaggtgggtgtaaaaggaggacgaggcttagctgc

SEQ ID NO: 93 - DNA, 483 bp, Artificial Sequence
  probe specific to S haplotype of Brassicaceae
ttcgacgatccatcaagcggggattactcgtacaagctcgaaccccgaaggcttcctgagttttatctattgctaggcgacgttcgtgagcatcggagcggtccatggaatggaatccaatttagtggaataccagaggaccaaaagttgagttacatggtgtacaatttcacagagaatagtgaggaggttgcttatacatttcgaatgaccaacaatagcttctactcgagattgacaataaattctgaagggtatttggagcgacagacgtgggctccgtcatcagtggtatggaacgtcttctggtcttctcctaaccatcagtgcgatatgtacaggatgtgtgggccttactcttactgtgacgtgaacacatcaccgtcatgtaactgtatccaagggttcaaacccgggaatgtgcagcagtgggctctgagaaaccaaataagtgggtgtaaaaggaggacgcggctgagctgc

SEQ ID NO: 94 - DNA, 486 bp, Artificial Sequence
  probe specific to S haplotype of Brassicaceae
tcagatgatccgtcaagcggggattacttgtacaagctcgaaccccgaaaacttccggagttttatctatggaatgaggactttccaatgcatcggagcggtccatggaatggagtcagatttagtggtataccagaggaccaaaagttgagctacttggtgtataatttcacagagaatagtgaagaggtggcttacacattccgaatgaccaacaacagcttttactcgagattgacagtaagttcctcagggtattttgagcgactgacgtggaatccgtcattagggatatggaacgtgttctggtcttctccagtggacttccattgcgacttgtacgtgagttgtgggccttactcttactgtgacgtgaacacatcacctgtgtgtaactgtatccaagggttcaatccctggaatatgcaggagtggaatctgagagtaccggcaggtgggtgtataaggaggaccaagcttagctgc

SEQ ID NO: 95 - DNA, 489 bp, Artificial Sequence
  probe specific to S haplotype of Brassicaceae
ttagatgatccgtcaagcggggattactcgtacaagctccaagcccgaagttatcctgagttttatctaattaaaaaaaaagtctttattgggcatcggagtggtccatggaatggaatccgatttagtgggataccagaggaccaaaagttgagttacatggtgtacaatttcacagagaatagagaggagatcgcttatacatttcgaatgaccaacaacagcttctactcgcgattgacaataagttccgaagggtattttgagcgtctgacgtggactctgtcatcaaatatgtggagcgttttctggtcttctccagtggacctccagtgcgatgtgtacaagtcttgtgggccttactcttactgtgacgtaaacacatcaccggtgtgtaactgtgtccaagggttctatcccaagaaccagcagcagtgggatgtgagagtcgcttccagtgggtgtataaggaggacgcgtcttagctgc

SEQ ID NO: 96 - DNA, 486 bp, Artificial Sequence
  probe specific to S haplotype of Brassicaceae
tcagatgatccatcaagcggggagttcttgtacgagctcgaaaccggaagacttcctgaattttatctatcgaaggggatctttccagcgtatcggagtggtccatggaatggaatccgatttagtggcataccagatgaccaaaagttgagttacctggtggacaatttcacagataatagtgaagaagtcacttatacattccgaatgaccaacaacagcatctactcgaagttgacagtaagtttctcagggtattttgagcgacagacgtggaatccgtcattagggatgtggaacatgttctgggcttttccaatggcctcacagtgcgatacgtacaggaggtgtgggccttactcttactgtgacgtgagcacatcaccgatttgtaactgtatccaagggttcaatccctcgaatgtgcagcagtgggatcagagatcctggtcaggtgggtgtataaggaggacgaggcttagctgc

SEQ ID NO: 97 - DNA, 492 bp, Artificial Sequence
  probe specific to S haplotype of Brassicaceae
tcagatgatccctcaagcggggaaatctcgtaccaactagacgctactccaagcggaatgtatgagttctatctattgaaaagcggctcacgagcccaccggagcggtccatggaatggagtccgatttagtggcataccaggggatcaagagttgagttacatggtgtacaatttcacagagaatagtgaggaggtttcttattcatttagaatgaccaacaacagcatctactcgatattgaaagtaagttccgatggggttttggagcgactgacctggaccccgaactcaattggatggaacttgttctggtatttaccattggaaaaccagtgcgatgtgtacatggtttgtgggcgttactcttactgtgacgtgaacacatcacccttgtgtaactgtatccaagggttcaatcgctcgaatgaggagcggtgggatctgaaagactggtcaagcgggtgtatgaggaggacgcagctgagctgc

SEQ ID NO: 98 - DNA, 486 bp, Artificial Sequence
  probe specific to S haplotype of Brassicaceae
tcagatgatccttcaagcgggaattactcgtacaagctcgaaacccgaaggcttcctgagttttatctatcgagtggagtttttcgattgcatcgtagtggtccgtggaatggaatccaaattagtggcataccagaggaccaaaatttgcattacatggtgtacaatttcatagagaatagtgaagaggtcgcttatacattccgaatgaccaacaacagcatctactcgagactgacactaggtttctcaggggactttcagcgactgacgtggaatccgtcaatagggatatggatcttgttctggtcttctccagtggacccccagtgcgatacatacgtaatgtgtgggcctaacgcttactgtgacgtgaacacatcaccggtatgtaactgtatccaagggttcaatccctggaatgtgcagctgtgggatcagagagtctgggcaggtgggtgtataagaaggacgcagcttagctgc

SEQ ID NO: 99 - DNA, 489 bp, Artificial Sequence
  probe specific to S haplotype of Brassicaceae
tcagatgatccctcaagcggggaaatctcgtactttctagacactgagagcggaatgcctgagttctatctattgaaaagcggcttacgagcctaccggagtggtccatggaatggagtccgatttagtggcataccaggggaccaatatttgagttacatggtgttcaatttcacagagaatagtgaggaggttgcttatacatttcgaatgaccacccacagcatctactcgagattgaaaataagttctgaagggtttttggagcgattgacgtggaccccgaactcaattcaatggaacttgttctggtatttaccagtggaaaaccagtgcgatgtgtacatggtttgtggggtttactcttactgtgacgagaacacatcaccggtgtgtaactgtatccaagggttcatgcccttgaatgagcagcgatgggatctgagagactggtcaagcgggtgcacaaggaggacgcggcttagctgc

SEQ ID NO: 100 - DNA, 489 bp, Artificial Sequence
  probe specific to S haplotype of Brassicaceae
tcagatgatccctcgaggggggaattctcgtaccaactagacactcaaaggggaatgcctgagttctttataatgaaagaaggctcacaaggccaacggagcggtccatggaatggagtccaatttagtggcataccagaggaccgaaagttgagttacatggtgtacaatttcacagagaataatgaggaggttgcttatacgtttcgagtgaccaacaacagcttttactcgagactgaaaataagtcccgaaggggttttagagcgattgacgaggaccccgacaacagttgcatggaacgtgttctggtctgtaccagtggatacccggtgcgatgtgtacatggcttgtgggccttacgcttactgtgacatgaatacatcaccgttgtgtaactgtatccaagggttcaagcgcttcaatgagcaggaatgggaaatgagagacgggtcaagtgggtgtataagggggacgcggctgagctgc

SEQ ID NO: 101 - DNA, 486 bp, Artificial Sequence
  probe specific to S haplotype of Brassicaceae
tttgatgatccgtcaagtggagaattctcgtacaagctcgaaacccgaaggcttcctgagttttatctattgaaaaacggctcaccaggtcagcggagcggtccatggaatggagttcaatttagtggcataccagaggaccaaaagttgagttacatggtgtacaatttcacagagaatagtgaggaggttgcttatacatttcgaatgaccgacaacagcatttactcgagaattcaaataagtcccgaagggcttttggagcgactgacgtggactccgacatcagggacatggaacttgttctggtctgcaccagtggatatccagtgcgatgtgtacatgacgtgtgggccttacgcttactgtgacgtgaacacatcaccggtgtgtaactgtatccaagggttcatcccctttgatatgcagcagtgggctctgagagacgggacaggtgggtgtataaggaggacgcggctgagctgc

SEQ ID NO: 102 - DNA, 486 bp, Artificial Sequence
  probe specific to S haplotype of Brassicaceae
ttaaatgatccctcaagcgggaattactcgtacaggctcgaaacccgaaggtttcctgaattttatctatggagtggggtctttatattgtatcggagtggtccatggaatggaatccgatttagtggcatactagaagaccaaaagttgagttacatggtgtacaatttcacagagaatagtgaagaagtcgcttatacatttcgaatgaccaacaacagcatgtatacgagattgacagtaagtttctcaggggattttgaacgacagacgtggaatccgtcaatagggatgtggaacaggttctgggcttttccattggactcacagtgcgatgcgtacacagcgtgtgggccttactcttactgtgacgtaaccacatcaccgatttgtaactgtatccaagggttcaatccctcgaatgtggagcagtgggatctgagaagctggtttggtgggtgtataaggaggacgcggcttagctgc

SEQ ID NO: 103 - DNA, 486 bp, Artificial Sequence
  probe specific to S haplotype of Brassicaceae
tcagatgatccgtcaagcggcgatttctcgtacaagctcgaagcccaaaggcttcctgagttttatctatcgagcggggtctttcgattgtatcggagcggtccatggaatggagtccgatttagtggtataccagatgaccaaaagttgagttacttggtgtacaatttcacagagaatagtgaggaagtcgcttatacattccgaatgaccaacagcagcatttactcgagattgatgctaagtttctcagggtatattgagcgacagacgtggaatccatcattaaggatgtggaacgtgttctggtcttttccattggactcacagtgcgattcgtaccggatgtgtgggcctaacgcttactgtgacgtgaacacatcaccgatttgtaactgtatccaagggttcaatccctcgaatgtgcagcagtgggatcagagagtctgggcaggtgggtgtataaggaggacgcggcttagctgc

SEQ ID NO: 104 - DNA, 489 bp, Artificial Sequence
  probe specific to S haplotype of Brassicaceae
tatgatgatccctcaagcggggaaatctcgtacaaactagacactgaaaggggattgccagagttttatctattgaaaaacggcttacgagcccaccggagcggtctatggaatggagtccaattttatggcataccagaggacctaaaattgagttacatggtgtacaactacacagagaatagtgaggaggtcgcttatacatttcgagtgaccaacaacagcatctattcgatattgaaagtaagttccggagagtttttggcgagactgactacgactccgtcatcatgggaatggagcttgttctggtattcaccagcggagccccagtgcgatgtgtacaagacttgtgggccttactcttactgtgacgtgaacacgtcaccggtgtgtaactgtatccaagggttcatgcccaggaatgtccagcagtgggagctgagaaacccgtcaggtgggtgtataaggaggacgcagctgagctgc

SEQ ID NO: 105 - DNA, 489 bp, Artificial Sequence
  probe specific to S haplotype of Brassicaceae
tcagatgatccctcaagcgggaatttctcgtacaagctcgaaaaccaaaggcttcctgagttttatctatcgagtcatggaatttttcgattgcatcgaagtggtccatggaatggaatcggatttagtggcataccagaggacgaaaagttgagttacatggtgtacaatttcacagagaatagtgaagaggttgcttatacattccgaatgaccaacaacagcatctattcgagattgacactaatttccaaaggggattttcagcgactgacgtgggatccgtcattagaaatatggaacatgttctggtcttctccagtggacccccagtgcgattcatacataatgtgtggggcgtacgcttactgtgacgtgaacacatcaccggtatgtaactgtatccaagggttcaacccccggaatatacagcggtgggatcagagagtctgggcaggtgggtgtgtaagaaggacgcggctaagctgc

SEQ ID NO: 106 - DNA, 486 bp, Artificial Sequence
  probe specific to S haplotype of Brassicaceae
tcagatgatccttcaagcggggattacttgtacaagctcgaaacccgaaggtttcctgaattttatctatcgagtggggtctttcgattgcataggagtggtccatggaatggaatccgatttagtggcatactagatgaccaaaagttgagttacttggcgtacaatttcacagagaatagtgaagaagtcgcttatacattccgaatgatcaacaacagcatctactcgagattgacagtaagtttctcagggtattttgaacgacagacatggaatccgtcattagggatgtggaacatgttctggtcttttccactggactcacagtgcgatggctacaggatgtgtgggccttacgcttactgtgacgcgaacacatcaccgatttgtaactgtatacaagggttcaatcccttagatgcggagcagtgggatctgagaagctggtcaggtgggtgtataaggaggacgcagcttagctgc

SEQ ID NO: 107 - DNA, 489 bp, Artificial Sequence
  probe specific to S haplotype of Brassicaceae
tcagatgatccgtcaagtggggaaatctcgtacttcctagacattcaaacgggaatgcctgagttctatctattgcaaagcggcgcacgaatgcaccgaagcggtccatggaatggagtccgatttagtggcatgccaggggaccaaaagttgaattacatggtgtacaatttcacagagaatagtgaggacgttgcttatacatttcgaatgaccaacaagagcatctactcgagattgaaagtaagttccgaagggtttttggagcgactgacgtggaccccaaattcaattacatggaacatgttctggtatttaccattggaaaaccagtgcgatatttacatgatttgtgggcgttacgcttactgtgacgtgaacacatcaccgttgtgtaactgtatccaagggttcaatcgctcgaatgaggagcggtgggatctgaaagattggtcaagcgggtgtataaggaggacgcggctgagttgc

SEQ ID NO: 108 - DNA, 486 bp, Artificial Sequence
  probe specific to S haplotype of Brassicaceae
tcagaggatccctcaagcggggaaatctcgtacaagctcgaaatgagaaggcttcctgagttttatctatggaatgaggactttccaatgcatcggagtggtccatggaatggaatcgaatttagtggaataccagaggaccaaaagtcgagttacatggcgtacaatttcacagagaatagtgaaggagtcgcttatacattccgaatgaccaacaacagcatctactcgagattgacagtaagttcagaagggaattttgagcgactgacgtggaatccgttattggggatgtggaacgtgttctggtcttctccagtggacgcccagtgcgatatgtacaggacgtgtgggccttactcttactgtgacgtgaatacatcgccggtttgtaactgtatccaagggttcaatccctcgaatgtgcagctgtgggatctgagagacggggcaggtgggtgcataaggaggacgcggctgagctgc

SEQ ID NO: 109 - DNA, 486 bp, Artificial Sequence
  probe specific to S haplotype of Brassicaceae
tcaaatgatccgtcaagcggcaatttctcgtacaagctcgaagcccaaaggcttcctgagttttatttatggaatgagaaatttccatggcatcggagtggtccatggaatggaatcgaatttagtggcataccagaagacaaagagttgagttacatggtgtacaatttcacagagaatagtgaagaggtcgcttatacattcctaatgaccaacaacagcatctactctagattgacaataaattccgcagggtattttcagcgactgacgtgggatccgttattaggaatgtggaacgtgttctggtcttctccagtggacctccagtgtgattcgtacaggaggtgtgggccttatgcttactgtgacgtgaccacatcaccggtttgtaactgtatccaaggattcaatcccaggtttgtggagcggtgggatatcagagactggtcagctgggtgtataaggaggacgcgtcttagctgc

SEQ ID NO: 110 - DNA, 486 bp, Artificial Sequence
  probe specific to S haplotype of Brassicaceae
ttaaatgatccttcaagcgggaatttctcgtacagactcgaaacccgaaggcttcctgagttttatctatcaaaacgcgacgttccagtgcatcggagcggtccatggaatggaatccgatttagtggcataccagaggacgaaaagttgagttacatggtgtacaatttcacagagaatagtgaggaggctgcttatacattcctaatgaccaacaacaacatctactcgagattgacaataagttcggatgggagttttcagcgactgacgtggactccgtcatcaggcgcgtggaacgtgttctggtcttctccagtaaaccccgagtgcgatttgtacatgatttgtgggcctgacgcttactgtgacgtgaacacatcaccgtcgtgtatctgtatccaagggttcaatcccaaggatctgccgcagtgggatctgagagactggacaagtgggtgcataaggaggacacggctgagctgc

SEQ ID NO: 111 - DNA, 489 bp, Artificial Sequence
  probe specific to S haplotype of Brassicaceae
tcagatgatccctcaagcggggaaacctcgtacaaactagacactcaaaggggtttgcctgagttctatctattgataaacggctcacgaggccaacggagcggtccatggaatggaatccgatttagtggcataccagaggacctaaggttgagttacatggtgtacaatttcatagagaatagtgaggaggtcgcttatacatttcgaatgaccaacaacagcatctactcgagattgaccataagttccgaagggcttttggagcgatggacgtgggccccggcatcattttcatggaacttgttctggtctttaccagcggatacctggtgcgatgtttacatggcttgtgggccgtacgcttactgtgacgtgaacacctcaccggagtgtaactgtatacaagggttcaaccgctccaatgagcagcagtgggatctgcgagacgggtcagctgggtgtgtaagggggacgcggctgagctgc

SEQ ID NO: 112 - DNA, 489 bp, Artificial Sequence
  probe specific to S haplotype of Brassicaceae
tcagatgatccctcaagcgggaatatcaagtaccaactagacactcaaaggggaatgcctgagttctatctattgaaagagggctctcgagcccaccggagtggtccatggaatggtgtccaattttatggcataccagaggaccaaaagttgagttacatggcgtataatttcatagagaatagtgaggaggttgcttatacatttcgaatgaccaacaacagcatctactcgagattgaaaataaattccgatgaatatttggatcgattgacgtggaccccgacatcaactgcatggaacttgttctggtctgcaccagtggatatccggtgcgatgtatacatggcttgtgggcctgacgcttactgtgacgtgagcacatcaccggtttgtaactgtatccaaggattcaagcgcagcgatgagcagcagtgggatctgagagacccgtcaagtgggtgtataagggggacgccgctgagctgc

SEQ ID NO: 113 - DNA, 486 bp, Artificial Sequence
  probe specific to S haplotype of Brassicaceae
tcagatgatccatcaagcggggaattctcgtacaagctcgaaccccgaaggcttcctgagttttatatatttattgaagacattccagtacatcggagtggtccatggaatggaatccgatttagtggcatactagaggaccaaaagttgagttacatggtgtacaatttcacagagaatagtgaagaggtcgcttatgcattccgaatgaccaacaacagcatctactcgagattgacattaagttccgaagggtattttcagcgactgacgtggactccatcgtcagtggtatggaacctgttctggtcttctccagcgaacgtcgagtgcgatttgtacagggtttgtgggcctaacggttactgtgacatgaacacatcaccgtcgtgtaactgtatccaagggttcaatccccgtaatatgcagcagtgggatctgagagatccgtcaagtgggtgtataaggaggacgttgctgagctgc

SEQ ID NO: 114 - DNA, 486 bp, Artificial Sequence
  probe specific to S haplotype of Brassicaceae
ttagatgatccatcaaacgggaatttctcgtacagccttgaaaaacgagagcttcctgagttttatctatataaaggcaactttcgagtgcatcggagtggtccatggaatggaatcgcatttagtggcataccagaagaccaaaagttgagttacatggtgtacaatttcatagagaatagtgatgaggccgcttatacattccgaatgaccaacagcagcatctactcgaaattgacaataaattcggaagggagatttcagcgactgacgtggactccatcatcaggcgcgtggaacgtgttctggtcttctccagagaaccccgagtgcgatttgtacatgatttgtgggccttacgcttactgtgacttgaacacatcaccgtcgtgtaactgtatccaagggttcaatcccggggatgtggagcagtgggatctgagagactggacaagtgggtgcataaggaggacacggctgagctgc

SEQ ID NO: 115 - DNA, 486 bp, Artificial Sequence
  probe specific to S haplotype of Brassicaceae
tcaaatgatccatcaagcggaaatttctcgtacaagctcgaaaaccgagagcttcctgagttttatctacagcaaaacgacattcgagcgcatcggagcggtccatggaatggtatcggatttagcgccataccagaggaccgaaagttgagttacatggtgtacaatttcacagagaatagtgaagaggtcgcttatacatttctaatgaccaacgacagcatttactcgagaattcaaatgagttccgaaggggatttgcggcgactgatgtggacgccgacatcatgggaatggagtttgttctggtctgcaccagtggatcctcagtgcgatgtgtacaagacatgtggaccttacgcttactgtgacctgaacacatcaccattgtgtaactgtatccaagggttcatgccctcgaatgtgcagcagtgggatctgagaaacccgtcaagtgggtgtataagaaggacgcggctgggctgc

SEQ ID NO: 116 - DNA, 489 bp, Artificial Sequence
  probe specific to S haplotype of Brassicaceae
tcagatgatccctcaagcgggaatttctcgtacaagctcgaaaaccaaaggcttcctgagttttatctatcgagtcatggaatttttcgattgcatcgaagtggtccatggaatggaatcggatttagtggcataccagaggacgaaaagttgagttacatggtgtacaatttcacagagaatagtgaagaggttgcttatacattccgaatgacaaacaacagcatctactcgagattgacactaagttccaaaggggattttcagcgactgacttgggatccgtcattagaaatatggaacatgttctggtcttctccagtggacccccagtgcgattcatacataatgtgtggggcgtacgcttactgtgacgtgaacacatcaccggtatgtaactgtatccaagggttcaatccccggaatatacagcggtgggatcagagagtctgggcaggtgggtgtgtaaggaggacgcagctgagctgc

SEQ ID NO: 117 - DNA, 486 bp, Artificial Sequence
  probe specific to S haplotype of Brassicaceae
tcggacgatccgtcaagcggggattactcgtacaagctcgaaccccgaaggcttcctgagttttatctactgcaaggagacgttcgagagcatcggagtggtccatggaacggaatccgatttagtgggatactagaggaccaaaagctgagttacatggagtacaatttcacagagactagtgaggaggtcgcttatacattccgaatgaccaacaacagcttctactcgagattgacactaagctccacagggtattttgagcgactgacgtgggctccgtcatcagtgatatggaacgtcttctggtcttctccagcaaacccccagtgcgatatgtacaggatgtgtgggccttactcttactgtgacgtgaacacatcaccatcgtgtaactgtatacaagggttcgatcccaggaatttgcagcagtgggctctgagaatctcattaagggggtgtaaaaggaggacgctgctgagctgc

SEQ ID NO: 118 - DNA, 489 bp, Artificial Sequence
  probe specific to S haplotype of Brassicaceae
tcagatgatccgtcaagcggggaattctcgtaccaactagacactcaaaggggaatgcctgagttcttagtattaaaagaaggctatccaggccaccggagcggtccatggaatggagtccgatttagtggcattccagaggaccaaaagttaagttacatggtgtacaatttcacagagaatagtgaggaggttgcttattcatttcgagtgaccaacaacagcatctactcgagactgaaaatcaattccgaagggtttttggagcgattgacgtggaccccagcatcaagtgcgtggaacttgttctggtctgtaccagtggatacccggtgcgatgtgtacatgtcttgtgggccttacgcttactgtgacgtgaacacatcaccggtttgtaactgtatccaagggttcaatcgctccaatgagcagcagtgggatatgagagacggggcaagtgggtgtataagggggacgcagctaagctgc

SEQ ID NO: 119 - DNA, 489 bp, Artificial Sequence
  probe specific to S haplotype of Brassicaceae
tcagatgatccatcaagcggggaaatctcgtacaagctcgaaccccgaagacttcctgagttttatctattgaaacgccgcgtctttcgactgcatcggagtggtccatggaatggaatccgatttagtggcataccagaggaccaaaagttgagttacatgatttacaatttcacagagaatagtgaagagctcgcttatacattccgaataaccaacaacagcatctactcgatattgacagtaagctccgaagggaaattagagcggctgatgtggaatccgtcattagcgatgtggaacgtgttctggttttttccagtggactctcagtgcgatacgtacatgatgtgtgggccttactcttactgtgacgtgaacacatcaccggtatgtaattgtatccaagggttcaatcccatgtacgtggaggagtgggatctgagagagtggtcaagtgggtgtataaggaggacgctgcttagctgc

SEQ ID NO: 120 - DNA, 489 bp, Artificial Sequence
  probe specific to S haplotype of Brassicaceae
tacgatgatccctcaagcggggaaatcgtgtacaaactagacactcaaaggggaatgcctgagttttttctattaaaaaatgactttccagcgcatcggagtggtccatggaatggtatcggatttagtggcctacctgaggaccataagttgggttacatggcgtacaatttcatagagaatagtgaagaggttgcttattcatttcgaatgaccaacaacagcatttactcgagattggaaataaattccgacggagatttggagcgactgatctggactccgacatcatgggaatggagcttgttctggtcttcaccagtggatctccagtgcgatgtgtacaagacttgtgggccttacgcttactgtgacttgaacacatcaccattgtgtaactgtatccaagggttcacgccctcgaatgtgcagcagtgggatctgagaaacccgtcagctgggtgtataaggaggacgaggctgagctgc

SEQ ID NO: 121 - DNA, 183 bp, Raphanus sativus
tttcaagaagctaatatgatggtgccatgtgacacaagtttccctgggggttgtggatccggaaataacggaaaaagggtctgcgagaggtcatatggggttgctaacaagccttctaattgcacatgtgtaccttttatagataataaccgactttgtaaatgtgtcgtaagattatgctaa

SEQ ID NO: 122 - DNA, 183 bp, Raphanus sativus
gctcaagaagtggaggctaatgtgctggggacttgtgactataccttccttttgggtggaccatgtggaaaatcaggaggagataaagcctgcgataagtcacgtactaataagaagaatccttatgattgcaaatgtgtacatacggtaccgaaaggaacatgttgttgtggtctgaaataa

SEQ ID NO: 123 - DNA, 178 bp, Raphanus sativus
ttatgtttcatattcatcgtttcagttaatattcaaggctttggtatgcgtggaaaatgtggcgattttggagccaagaaatgcgaaagcttatatttggctgataagggcaagaagccttcagggtgcaaatgcacagattctccaaataataattatgtttgtgattgtaaatagc

SEQ ID NO: 124 - DNA, 186 bp, Raphanus sativus
gttcaagatgtggaagctaatctgatgaagcggtgcacccatcagttaccttttcgcggaacatgtggcagttcaggagacgaggtctgcaaaaaattatattcggctgaaacgaagacgaatccttctcgttgcgaatgtatacctgattataaaaatcgattttgtcgttgtaaactgtgctaa

SEQ ID NO: 125 - DNA, 204 bp, Raphanus sativus
gtgcaggctaatctcatgaacaagtgtaccgactacataaatttgcttggacgatgtgaaagttcaggtgacgagctctgcgcgagttcatatgagagcaataagtatacgaagcctcataattgcgaatgtaaagatgtgaaaacgaagattcagaacaataaagatgttatacgtggacgctgtcgttgtgtactgtgttaa

SEQ ID NO: 126 - DNA, 171 bp, Raphanus sativus
catgaagtggaagctaataagatcaagatgtgtcctgatcccatcaaattgagaggaaaatgtagcgaatcaggagggcttgccgcctgcgcaaaatcatttaaaaggaagaatactgctggttgctcatgtgatgatcatgatgaaaaaggaagttgttgttgtttgtaa

SEQ ID NO: 127 - DNA, 199 bp, Raphanus sativus
tgttcaaggagtgggagctaatctgaggaaaacatgcgttcatagattaaatacgggtggaagttgtggcaaatcaggacagcatgactgcgaagcctactatacgaataaaacgaaaaaacaggctttctattgcaactgtacaagtccttttcgaactcgatattgtgattgtgcggttaaatgcaaatatggatag

SEQ ID NO: 128 - DNA, 171 bp, Artificial Sequence
  probe specific to S haplotype of Raphanus sativus
aatatgatggtgccatgtgacacaagtttccctgggggttgtggatccggaaataacggaaaaagggtctgcgagaggtcatatggggttgctaacaagccttctaattgcacatgtgtaccttttatagataataaccgactttgtaaatgtgtcgtaagattatgctaa

SEQ ID NO: 129 - DNA, 165 bp, Artificial Sequence
  probe specific to S haplotype of Raphanus sativus
aatgtgctggggacttgtgactataccttccttttgggtggaccatgtggaaaatcaggaggagataaagcctgcgataagtcacgtactaataagaagaatccttatgattgcaaatgtgtacatacggtaccgaaaggaacatgttgttgtggtctgaaataa

SEQ ID NO: 130 - DNA, 147 bp, Artificial Sequence
  probe specific to S haplotype of Raphanus sativus
attcaaggctttggtatgcgtggaaaatgtggcgattttggagccaagaaatgcgaaagcttatatttggctgataagggcaagaagccttcagggtgcaaatgcacagattctccaaataataattatgtttgtgattgtaaatag

SEQ ID NO: 131 - DNA, 168 bp, Artificial Sequence
  probe specific to S haplotype of Raphanus sativus
aatctgatgaagcggtgcacccatcagttaccttttcgtggaacatgtggcagttcaggagacgaggtctgcaaaaaattatattcggctgaaacgaagacgaatccttctcgttgcgaatgtatacctgattataaaaatcgattttgtcgttgtaaactgtgctaa

SEQ ID NO: 132 - DNA, 195 bp, Artificial Sequence
  probe specific to S haplotype of Raphanus sativus
aatctcatgaacaagtgtaccgactacataaatttgcttggacgatgtgaaagttcaggtgacgagctctgcgcgagttcatatgagagcaataagtatacgaagcctcataattgcgaatgtaaagatgtgaaaacgaagattcagaacaataaagatgttatacgtggacgctgtcgttgtgtactgtgttaa

SEQ ID NO: 133 - DNA, 156 bp, Artificial Sequence
  probe specific to S haplotype of Raphanus sativus
aataagatcaagatgtgtcctgatcccatcaaattgagaggaaaatgtagcgaatcaggagggcttgccgcctgcgcaaaatcatttaaaaggaagaatactgctggttgctcatgtgatgatcatgatgaaaaaggaagttgttgttgtttgtaa

SEQ ID NO: 134 - DNA, 180 bp, Artificial Sequence
  probe specific to S haplotype of Raphanus sativus
aatctgaggaaaacatgcgttcatagattaaatacgggtggaagttgtggcaaatcaggacagcatgactgcgaagcctactatacgaataaaacgaaaaaacaggctttctattgcaactgtacaagtccttttcgaactcgatattgtgattgtgcggttaaatgcaaatatggatag

(19) United States
(12) Patent Application Publication
(10) Pub. No.: US 2004/0091888 A1
(43) Pub. Date: May 13, 2004

(54) Method for identification of S genotype in brassicaceae

(76) Inventors: Takeshi Nishio, Miyagi (JP); Ryo Fujimoto, Miyagi (JP); Toyokazu Akamatsu, Shizuoka (JP); Koji Sakamoto, Shiga (JP); Hideaki Hanzawa, Gunma (JP); Shunsuke Okamoto, Miyagi (JP)

Correspondence Address: FISH & RICHARDSON P.C., 45 ROCKEFELLER PLAZA, SUITE 2800, NEW YORK, NY, 10111, US

(21) Appl. No.: 10/392,536

(22) Filed: Mar. 20, 2003

(30) Foreign Application Priority Data
Mar. 20, 2002 (JP) 079350/2002

(51) Int. Cl. C12Q 1/68

(52) U.S. Cl. 435/6

(57) ABSTRACT

The present invention relates to novel S-haplotype specific DNA fragments of plants belonging to Brassicaceae, and a method for identifying S-haplotypes of plants belonging to Brassicaceae.

### BACKGROUND OF THE INVENTION

1. Technical Field

The present invention relates to novel S-haplotype specific DNA fragments of plants belonging to Brassicaceae, and a method for identifying S-haplotypes of plants belonging to Brassicaceae using the novel S-haplotype specific DNA fragments. More particularly, the present invention relates to a method for simply performing line selection in the breeding of plants belonging to Brassicaceae and verification of seed purity of hybrid cultivars or parent lines by identifying the S-haplotypes of plants belonging to Brassicaceae.

2. Background Art

Plants belonging to Brassicaceae possess “self-incompatibility,” in which self-pollination does not result in fertilization nor seed-setting. Self-incompatibility can also be said to be an important mechanism in maintaining species diversity, because it inhibits inbreeding by recognizing a plant's own pollen and thereby specifically inhibiting pollen germination and pollen tube elongation.

In vegetables belonging to Brassicaceae, such as cabbage, cauliflower, and broccoli, F₁ hybrid cultivars with high homogeneity and high productivity are actively produced using the self-incompatibility. However, breeding of a more homogenous F₁ hybrid cultivar requires information concerning the self-incompatibility-related S-gene of a parent line (JP Patent Publication (Kokai) No. 8-275779).

S-gene is a multiple allele that regulates self-incompatibility, and Brassicaceae is known to have many haplotypes (S-haplotypes) therein. Generally, when the S-haplotype of a pollen and that of a pistil are matched, self-incompatibility is induced. Since S-haplotypes are not expressed in appearance as phenotypes, conventionally S-haplotypes could only be determined by crossing and examining the number of the resulting seeds. However, there are as many as about 50 S-haplotypes in the same species, so that the identification of S-haplotype using this method is not easy.

Hence, methods for analyzing S-locus glycoprotein, which is an S-gene product and is expressed in the stigma of a pistil by isoelectric focusing or an immunochemical technique, were developed for the first time (Nishio, T and Hinata K. (1980) Euphytica 29: pp. 217-221, Hinata, K. and Nishio, T. (1981) Theor. Appl. Genet. 60: pp. 281-283, Nou, I. S. et al, (1993) Sex. Plant Rep. 6: pp 79-86, Ruffio-Chable, V. et al, (1997) Theor. Appl. Genet. 94: pp. 338-346, Okazaki, K. et al, (1999) Theor. Appl. Genet. 98: pp. 1329-1334). In these methods, 5 to 10 stigmas are collected, soluble proteins are extracted with phosphate buffer solution or the like, and then isoelectric focusing is performed. Concerning the detection of S-locus glycoproteins, since S-locus glycoproteins bind to concanavalin A, a method that detects concanavalin A labeled with FITC (fluorescein isothiocyanate) was reported for the first time (Nishio, T and Hinata K. (1980) Euphytica 29: pp. 217-221). This method was improved into one that involves binding of S-locus glycoproteins with concanavalin A, and then with peroxidase, followed by detection of peroxidase activity (Hinata, K. and Nishio, T. (1981) Theor. Appl. Genet. 60: pp. 281-283). However, glycoproteins other than S-locus glycoproteins are detected by the use of concanavalin A. Accordingly, detection became to be performed using an antibody to S-locus glycoproteins (Nou, I. S. et al, (1993) Sex. Plant Rep. 6: pp. 79-86), improving the accuracy of the S-haplotype identification method.

However, these methods have the following problems:

(1) Since stigmas are necessary as materials, it is required to cultivate plants to flowering for the identification of S-haplotype.

(2) Since isoelectric focusing is utilized, analysis is slightly expensive, and skill in analytical techniques is also required. When analysis is performed using antibodies, purification of S-locus glycoproteins to be used as an antigen and production of antibodies become necessary. Further, costs and skill in techniques also become necessary.

(3) There is a plurality of S-haplotypes having no S-locus glycoproteins (Okazaki, K. et al, (1999) Theor. Appl. Genet. 98: pp. 1329-1334). The method cannot be applied for the analysis of these S-haplotypes.

(4) Even when analysis is performed using antibodies, proteins other than S-locus glycoproteins may be detected (Nou, I. S. et al, (1993) Sex. Plant Rep. 6: pp. 79-86, Okazaki, K. et al, (1999) Theor. Appl. Genet. 98: pp. 1329-1334).

Next, methods that analyze S-gene DNA by Southern blot analysis were developed Nasrallah, J. B. et al, (1985) A, Nature 318: pp. 263-267, Sakamoto, K. et al, (1998) Mol. Gen. Genet. 258: pp. 397-403, (Okazaki, K. et al, (1999) Theor. Appl. Genet. 98: pp. 1329-1334, Kusaba, M. et al, (2000) Genetics 154: pp. 413-420). However, these methods have the following problems:

(1) Analysis can be performed using DNA extracted from leaves, so that analysis at a stage before flowering is possible. However, since relatively a large amount of DNA is required, it takes time to extract such an amount of DNA.

(2) The Southern blot analysis of plant genomic DNA requires a certain degree of skill in analytical techniques and is expensive.

(3) There are lines of the same S-haplotype but showing different band patterns (Kusaba, M. et al, (2000) Genetics 154: pp. 413-420).

Further, methods that analyze S-gene DNA by PCR-RFLP (Brace, J. et al, (1993) Sex. Plant Reprod. 6: pp. 133-138, Nishio, T. et al, (1994) Plant Cell Rep. 13: pp. 546-550, Brace, J. et al, (1994) Sex. Plant Reprod. 7: pp. 203-208, Nishio, T. et al, (1996) Theor. Appl. Genet. 92: pp. 388-394, Nishio, T. et al, (1997) Theor. Appl. Genet. 95: pp. 335-342, Sakamoto, K. et al, (2000) Plant Cell Rep. 19: pp. 400-406, Lim, S.-H. et al, (2002) Theor. Appl. Genet. 104: pp. 1253-1262, were also developed. However, these methods have the following problems:

(1) Genes other than S-locus glycoprotein gene (SLG) are also amplified by primers, so that it is difficult to identify S-haplotypes (Brace, J. et al, (1993) Sex. Plant Reprod. 6: pp. 133-138), or even when analytical accuracy is high, not all SLG alleles can be amplified since primers often have too high specificity to amplify all SLG (Nishio, T. et al, (1996) Theor. Appl. Genet. 92: pp. 388-394).

(2) Primers to amplify SRK cannot always recognize all SRK alleles (Nishio, T. et al, (1997) Theor. Appl. Genet. 95: pp. 335-342). Thus, there is a need to identify the S-haplotypes by combining analytical results using several primers.

(3) There are S-haplotypes lacking SLG(Okazaki, K. et al, (1999) Theor. Appl. Genet. 98: pp. 1329-1334, Sato, K. et al, (2002) Genetics 162: pp. 931-940).

It is very difficult to distinguish the cultivars by their seed appearance. Therefore, for the purpose of quality control of commercial seeds, one needs to check the contamination and/or outcross with other cultivars and/or mistaking a certain cultivar from another one. Those inspections include growout test wherein ecological and morphological properties are investigated, and biochemical methods that analyze proteins and nucleic acids.

The former method includes juvenile growout (distinguish by morphological characteristics at early stage of growth) and market stage growout (distinguish by morphological characteristics at marketable stage such as fruits) and the like. The latter method generally includes electrophoretic analysis of seed proteins, isozymes analysis (analyze the enzyme activity by electrophoresis and staining), nucleic acid analysis using the PCR method and the like.

However, the growout test is time-consuming, has poor reproducibility, and is labor-intensive. In addition, the biochemical techniques make assumptions based on the strength of the correlation between a specific phenotype of the cultivar and the band pattern of electrophoresis, and do not directly target a specific gene of the cultivar, so that difficulty in obtaining a conclusive proof is a problem within this technique.

Recently, “S-locus” has been shown to be a complex gene locus of the S-locus receptor kinase gene (SRK), S-locus glycoprotein gene (SLG) and an S-locus cysteine-rich protein gene (also referred to as SP11 or SCR) (Suzuki G et al. (1999) Genetics 153, pp. 391-400). When we studied the intra-species mutation for the nucleotide sequences of these genes, we found that SRK and its homologous gene, SLG, have 3 hypervariable regions (Nishio T & Kusaba M, (2000) Annals of Botany 85 Suppl. A, pp. 141-146), and the regions of SP11, except the signal peptide region, are very rich in mutation (Sato, K. et al, (2002) Genetics 162: pp. 931-940).

### SUMMARY OF THE INVENTION

We have considered that specific detection of target S-haplotypes becomes possible by performing the Southern blot analysis or the like using as probes these regions, which are present in S-locus and rich in mutation.

Thus, an object of the present invention is to provide S-haplotype specific DNA fragments, in particular a simple and highly accurate identification method of S-gene using the fragments that are present on SP11 and SRK genes.

As a result of thorough studies on the above problems, we have succeeded in identifying several types of novel S-haplotype DNA fragments and preparing probes that can specifically detect these DNA fragments. Further, it was found that since these probes have high specificity and do not bind to other DNA fragments on the genome of plants belonging to Brassicaceae, they are extremely effective for identifying S-haplotypes. Furthermore, we have found that detection using a dot blot method makes it possible to identify S-haplotypes more simply and rapidly, so that we have completed the present invention.

That is, the present invention relates to the following (1) to (20):

(1) a DNA fragment, which consists of a nucleotide sequence contained in at least a gene selected from the group consisting of an S-locus cysteine-rich protein gene, an S-locus receptor kinase gene and an S-locus glycoprotein gene that are present on the S-locus of plants belonging to Brassicaceae, and with which S-haplotypes can be specified;

(2) a DNA fragment, which consists of a nucleotide sequence contained in an S-locus cysteine-rich protein gene, and/or an S-locus receptor kinase gene that is present on the S-locus of plants belonging to Brassicaceae and with which S-haplotypes can be specified;

(3) a DNA fragment, which is defined by any one of the sequences selected from the group consisting of SEQ ID NOS: 1 to 17, SEQ ID NOS: 47 to 83, and SEQ ID NOS: 121 to 127, and with which S-haplotypes can be specified;

(4) a method for identifying S-haplotypes, which comprises detecting DNA fragments with which S-haplotypes can be specified from a plant or a group of plants belonging to Brassicaceae;

(5) the method of (4) above, wherein the DNA fragment, with which S-haplotypes can be specified consists of a nucleotide sequence contained in at least a gene selected from the group consisting of an S-locus cysteine-rich protein gene, an S-locus receptor kinase gene and an S-locus glycoprotein gene that are present on the S-locus of plants belonging to Brassicaceae;

(6) the method of (4) above, wherein the DNA fragment, with which S-haplotypes can be specified, consists of a nucleotide sequence contained in an S-locus cysteine-rich protein gene and/or an S-locus receptor kinase gene that are present on the S-locus of plants belonging to Brassicaceae;

(7) the method of (4) above, wherein the DNA fragment, with which S-haplotypes can be specified, is defined by any one of the sequences selected from the group consisting of SEQ ID NOS: 1 to 17, SEQ ID NOS: 47 to 83, and SEQ ID NOS: 121 to 127;

(8) a method of purity verification or quality control for seeds, which uses the method of (5) above;

(9) a method for breeding plants, which uses the method of (5) above;

(10) a plant or a cultivar, which is produced by the breeding method of (9) above;

(11) an oligonucleotide primer, which is for specifically amplifying the DNA fragment of (1) above, and has sequential 10 to 50 nucleotides in length.

(12) a probe, which hybridizes specifically to the DNA fragment of (1) above, and is for detecting the fragment;

(13) a probe, which is for detecting an S-haplotype specific DNA fragment which is defined by any one of the sequences selected from the group consisting of SEQ ID NOS: 18 to 34, SEQ ID NOS: 84 to 120, and SEQ ID NOS: 128 to 134;

(14) the method of (5) above, which is performed using a probe defined by any one of the sequences selected from the group consisting of SEQ ID NOS: 18 to 34, SEQ ID NOS: 84 to 120, and SEQ ID NOS: 128 to 134;

(15) a method for identifying S-haplotypes, which comprises the following steps of:

1) extracting DNA samples from a plant,

2) immobilizing the above DNA samples onto a support,

3) labeling probes for detecting S-haplotype specific DNA fragments, and hybridizing the probes to the above DNA samples, and

4) identifying the S-haplotypes of the plant based on the above labels.

(16) a method for identifying S-haplotypes, which comprises the following steps of:

1) immobilizing probes for detecting S-haplotype specific DNA fragments onto a support,

2) extracting and labeling DNA samples from a plant,

3) hybridizing the above labeled DNA samples to the probes on the support, and

4) identifying the S-haplotypes of the plant based on the above labels.

(17) a support for identifying S-haplotypes, on which the probe of (12) above is immobilized;

(18) a support for identifying the S-haplotypes, on which the probe of (13) above is immobilized;

(19) the support for identifying the S-haplotypes of (17) above, wherein any one support selected from the group consisting of a membrane, a glass plate, a capillary and a bead is used as the support;

(20) a kit for identifying the S-haplotypes, which comprises at least one or more elements selected from the following 1) to 3):

1) an oligonucleotide primer, which is for specifically amplifying the DNA fragment of (1) above, and has sequential 10 to 50 nucleotides in length,

2) a probe, which hybridizes specifically to the DNA fragment of (1) above, and is for detecting the fragment; and

3) a support, which has probes immobilized thereon that specifically hybridize to the DNA fragments of (1) above, and is for detecting the fragments.

The present invention is hereinafter described in more detail.

In this specification, “DNA” includes not only double-stranded DNAs, but also single-stranded DNAs composing the DNA. Further, a “DNA fragment” refers not only to a full-length DNA, but also to a partial DNA composing the DNA.

1. S-haplotype Specific DNA Fragments

The DNA fragments according to the present invention are S-haplotype specific DNA fragments, which are present on S-multiple allele loci (S-gene loci) of the genome of plants belonging to Brassicaceae. The DNA fragments comprise a nucleotide sequence specific to each S-haplotype, and can be used for specifying the S-haplotype of the plant belonging to Brassicaceae.

Further, in this specification, “S-haplotype(s)” refers to a cultivar of haplotypes, which are present on the above S-locus that are rich in intra-species variation.

The S-locus contains the above S-locus receptor kinase gene (SRK), S-locus glycoprotein gene (SLG), and S-locus cysteine-rich protein gene (may also referred to as SP11, or SCR). The DNA fragments of the present invention can be specified through PCR amplification of highly specific regions of the above genes located in the S-locus (in SP11, coding regions other than the signal peptide region) from genomic DNA extracted from plants belonging to Brassicaceae, and then sequencing and comparing the obtained DNAs.

For example, anthers are collected from B. oleracea plants having different S-haplotypes, and then the mRNA is isolated by a known conventional method. Next, using the above mRNA, a single-stranded cDNA is prepared, and then a SP11 cDNA is specifically amplified by PCR using the single-stranded cDNA as a template and primers prepared based on known S-genes or the like. The PCR products are cloned by a conventional method (for example, using a commercially available TA cloning Kit (Invitrogen) or the like), and then the nucleotide sequence is determined by a DNA sequencer.

Examples of the thus determined DNA fragments include DNA fragments having nucleotide sequences represented by SEQ ID NOS: 1 to 17, SEQ ID NOS: 47 to 83, and SEQ ID NOS: 121 to 127

2. Primers for Amplifying S-haplotype Specific DNA Fragments

The primers according to the present invention are oligonucleotide primers for specifically amplifying by PCR the above S-haplotype specific DNA fragments from the genomic DNA of plants belonging to Brassicaceae, and can be prepared using a conventional, such as a method using a commercially available software or the like for designing primers based on the sequences of the DNA fragments. The above primer is preferably 15 to 50 nucleotides in length, and in particular, 15 to 25 nucleotides in length. Examples of the primers can include, in the case of SP11, oligonucleotides that are defined by SEQ ID NOS: 35 to 46.

3. Probe for Detecting S-haplotype Specific DNA Fragments

The probe according to the present invention specifically hybridizes to the above S-haplotype specific DNA fragment, and is for detecting the DNA fragment. The length of the above probe is not specifically limited, and is preferably 100 to 300 nucleotides in length, and more preferably, 150 to 220 nucleotides in length. The probe may also be labeled with an isotope, enzyme, fluorescent substance, digoxigenin (DIG) or the like.

The above probe can be prepared from among the S-genes of the present invention based on the sequence of a region having particularly a high specificity to the S-haplotype. Examples of the probe include, in the case of SP11, nucleotide sequences that contain the nucleotide sequences defined by SEQ ID NOS: 18 to 34 and SEQ ID NOS: 128 to 134, and in the case of SRK, nucleotide sequences that contain the nucleotide sequences defined by SEQ ID NOS: 84 to 120.

4. Identification Method of S-haplotypes

The identification method of S-haplotypes according to the present invention comprises detecting S-haplotype specific DNA fragments from the genomic DNA of plants belonging to Brassicaceae, and then identifying the S-haplotypes. Examples of the S-haplotype specific DNA fragment include DNA fragments defined by SEQ ID NOS: 1 to 17, SEQ ID NOS: 47 to 83, and SEQ ID NOS: 121 to 127 described in 1. The present invention can be subjected to all Brassicaceae regardless of genus. As such an example, the methods for identifying the S-haplotypes of Brassica oleracea and Raphanus sativus are described in the examples of the present specification.

The above detection methods are not specifically limited, and any nucleic acid hybridization method using immobilized samples, such as the conventional Southern blot method, dot blot method, microarray, and gene chip can be used. In particular, the dot blot method is preferably used.

For example, the method for identifying the S-haplotypes of the present invention can be performed by the following steps of:

1) extracting DNA samples from plants;

2) immobilizing the above DNA samples onto a support;

3) labeling probes for detecting S-haplotype specific DNA fragments, and hybridizing the probes to the above DNA samples; and

4) identifying the S-haplotypes of the plant based on the above labels.

Further, the method for identifying the S-haplotypes of the present invention can be performed by the following steps of:

1) immobilizing probes for detecting S-haplotype specific DNA fragments onto a support;

2) extracting and labeling DNA samples from a plant;

3) hybridizing the above labeled DNA samples to the probes on the support; and

4) identifying the S-haplotypes of the plant based on the above labels.

Here, “DNA samples” may be prepared according to conventinal methods, for example by disrupting the leaves, seeds or the like of plants, and then extracting with an appropriate extract buffer solution. Further, a “support”, onto which DNA samples or probes are immobilized, is not specifically limited. For example, a membrane (for example, a nylon membrane), glass plate, capillary, bead (for example, glass beads) and the like can be used. Immobilization of DNA samples or probes onto the support is also not specifically limited, and can be performed according to conventional methods. Particularly in the case of probes, in addition to a method which immobilizes previously prepared probes onto a support, a method which synthesizes probes on a support may also be employed.

The above “probes for detecting S-haplotype specific DNA fragments” are nucleotides for specifically detecting the S-haplotype specific DNA fragments of the present invention as described in 3. Examples of such a probe can include polynucleotides, which contain the nucleotide sequences defined by SEQ ID NOS: 18 to 34, SEQ ID NOS: 84 to 120, and SEQ ID NOS: 128 to 134. As “labels” for DNA samples or probes, any known labels, such as an isotope label, enzyme label, fluorescent label, and digoxigenin (DIG) label can be used.

Further, the identification method of S-haplotypes of the present invention is superior to any conventional identification method of S-haplotypes in respect of the points mentioned below.

(1) A number of samples can be easily analyzed because electrophoresis is not used in the method of the present invention.

(2) With conventional electrophoresis, distinguishing is performed based on band patterns, so that S-haplotypes having different band patterns can be easily distinguished, but those having similar band patterns cannot be easily identified. In contrast, with the method of the present invention, all the S-haplotypes for which probes are available can be identified.

(3) The cost required per sample is low.

As a preferred embodiment of the identification method of S-haplotypes of the present invention, a method using the dot blot method is described below. The dot blot method comprises denaturing and immobilizing DNA or RNA samples onto an appropriate membrane, hybridizing specific probes thereto, and then qualitatively determining or identifying specific sequences in the sample. This method has an advantage such that there is no need to use a plant DNA sample with a high purification degree as required by the conventional Southern blot method and the PCR-RFLP method. For example, a DNA sample to be used herein can be prepared simply by crushing plant tissues (eaves or seeds) with extra buffer solution, extracting DNA at a high temperature, such as approximately 60° C., directly adhering the supernatant of centrifuged extract to a membrane, and then denaturing the DNA with alkaline solution.

The raw material and size of the membrane to be used in the dot blot method of the present invention are not specifically limited, and can be selected appropriately depending on the purpose. In particular, a nylon-made membrane is preferred.

The method for adhering DNAs to a membrane is not specifically limited. DNA may be adhered one by one using a pipette, or may be adhered using a commercially available 96-well dot blotting device. It is more efficient to use an instrument which adheres each DNA sample to the tip of a pin, each pin of its own separately so as to transfer the DNA sample to a membrane. However, to improve detection accuracy, it is essential to evenly distribute the amount of DNA to be adhered. To do this, the ratio of the amount of a plant tissue to that of an extract buffer solution must be kept at a constant level, and the degree of crushing and the fluid amount to be transferred to a membrane are also kept at a constant level.

For example, in an embodiment, 96 samples, or 384 samples of plant DNA are adhered to a 8 cm×12 cm nylon membrane, and then S-haplotype specific DNA fragments are detected using as probes highly specific regions of the SP11 gene, so that the S-haplotypes can be identified.

In the dot blot method, in order to detect hybridization of the probe, it is normally required that each probe must be labeled by any method. The above labeling method is not specifically limited, and any known labeling method, such as an isotope labeling, enzyme labeling, or fluorescent labeling may be used. In particular, digoxigenin (DIG) labeling is preferred. With DIG-labeled probes, the position of a sample to which each probe is bound can be detected using anti-DIG antibody-alkaline phosphatase complex.

The above dot blot method which adheres the DNA sample of a plant to a membrane can be performed well, when certain preliminary information on the S-haplotypes of the plant is available. However, when there is no such preliminary information, the method requires the analysis of existing intra-species S-haplotypes (approximately 50 in Brassicaceae) as probes in sequence, and this makes the process complicated. In such a case, it is preferable to use a method, which dots to a membrane the probes of the present invention that are capable of specifically detecting S-haplotype DNA fragments instead of the DNA samples of plant.

In a method for immobilizing the above probes, the genomic DNA of a plant, which is used as a sample, is labeled instead of probes, and then the S-haplotypes are determined from the positions of a membrane to which the plant DNAs hybridize. The plant DNA is labeled, for example, by PCR amplification using, as a substrate, a deoxinucleotide labeled with digoxigenin, isotope or the like, and the primers of the present invention. In this case, a plant DNA to be used as a template preferably has a certain high purification degree, as a result of purification by CTAB (Cetyl trimethyl ammonium bromide) method or the like.

The number and types of primers to be used in the above PCR amplification are not specifically limited, and can be appropriately selected according to the purpose. However, since not all of many S-haplotypes existing in the belonging species only a pair (forward and reverse) of primers relating to a specific nucleotide sequence are used, a mixed use of primers of a plurality of nucleotide sequences is preferred.

5. Support and Kit for Identifying S-haplotypes

The present invention also provides a support, which is used in the identification method of S-haplotypes. The support is prepared by immobilizing the above probes of the present invention on an appropriate support. The support, to which the probes are immobilized, is not specifically limited. In particular, a membrane (for example, nylon membrane), a glass plate, capillary, bead (for example, glass beads) are preferred. The method for immobilization of DNA samples or probes onto the support is not specifically limited, and can be performed according to a conventional method. Particularly in the case of probes, it may also be synthesized on a support, in addition to be immobilized onto a support after previously preparing..

For example, a microarray having a glass plate as a support can be prepared based on a conventional method, which involves, for example, aligning and adhering the probes of the present invention on a glass plate, using commercially available systems for preparing DNA microarray (New Genetic Engineering Handbook, YODOSHA, p280-284, (2000)).

Preferably, probes for SP11 and probes for SRK derived from the same S-haplotype are aligned such that the two types of probes correspond to each other, and binding to the both types of probes is used as an indicator, so that accuracy in identification of S-haplotypes can be improved.

Moreover, the present invention provides a kit for identifying S-haplotypes, which contains at least one or more elements selected from the above primers for amplifying S-haplotype specific DNA fragments and the probes for detecting the fragment, and the support for identifying S-haplotypes. In addition to the above essential elements, such a kit may contain other reagents and the like required for the identification method.

6. Use of Identification Method of S-haplotypes

The identification method of S-haplotypes of the present invention can also be used as a method of verification of seed purity and a method of quality control for seeds, or a method for plant breeding.

In plant breeding, possible applications of the method include, for example, a method which involves identifying S-haplotypes at the seedling stage, also in case of after selection, distinguishing S-haplotypes before crossing (including confirmation of homozygote or heterozygote). These applications make possible to reduced cultivation area and labor required for breeding, reduced breeding period, and the like. Further, also for a useful gene and/or deleterious gene existing near by S-locus, S-haplotypes are identified at an extremely early stage of cultivation, and thus plants having these genes are able to be selected, so that efficiency of breeding can be promoted. Moreover, the seed purity of parent lines can also be verified by examining the S-haplotypes of F₂ generation.

Further, according to the identification method of S-haplotypes of the present invention, during the period from seeds production to sales of seeds and/or seedlings, that can be examined more surely than conventional methods, without conducting cultivation tests, such as the contamination rate of commercial seeds that should be uniform, with other cultivar seeds, the possibility to mistake a certain cultivar for another one, and the like. Therefore, the identification method of S-haplotypes of the present invention can be applied to a method of verification of seed purity and a method of quality control of breeding.

### BRIEF DESCRIPTION OF DRAWINGS

FIG. 1 shows the results of dot blotting in Example 2.

FIG. 2 shows the results of dot blotting by double-sided labeling in Example 3.

FIG. 3 shows the results of dot (blotting by single-sided labeling) in Example 3.

FIG. 4 shows the result of Southern blotting in Example 4 using SP11-57 cDNA as a probe. In FIG. 4, the arrow denotes the position of a sample well.

FIG. 5 shows the results of identifying S-haplotypes (dot blot method) in Example 5 using SP11* probe and SP11 probe. [In FIG. 5: (a) SP11-25 probe, (b) SP11-32 probe, (c) SP11-12* probe, and (d) SP11-32* probe]

FIG. 6 shows the results of verifying the purity of parent lines in Example 6 by identifying (dot blot method) the S-haplotypes of F₂ generation using SP11* probe. [In FIG. 6: (a) SP11-18* probe, (b) SP11-39* probe; each number denotes an plant number.]

FIG. 7 shows the results of detecting S⁷ homozygous plant and S¹⁸/S³⁹ heterozygous plant in Example 7 using SP11* probe. [In FIG. 7, (a) S⁷ homozygous plant, and (b) S¹⁸/S¹³ heterozygous plant]

FIG. 8 shows the results of distinguishing B. oleracea plants in Example 8 using SP11* probe. [In FIG. 8, A (left) SP11-18* probe, and (right) SP11-15* probe; B schematically shows the blot method, and each number denotes a plant number.]

FIG. 9 shows the results of identifying S-haplotypes (dot blot method) of Raphanus sativus genomic DNA in Example 10.

This specification includes part or all of the contents as disclosed in the specification of Japanese Patent Application No. 2002-079350, which is a priority document of the present application.

### BEST MODE FOR CARRYING OUT THE INVENTION

The present invention is more specifically described by the following Examples. However, these examples are not intended to limit the present invention.

### EXAMPLE 1

Specification of S-haplotype Specific SP11 and SRK DNA Fragments of i B. oleracea

1. Isolation of SP11 and SRK from B. oleracea

Anthers were collected from B oleracea plants having different S-haplotypes, and then mRNAs were isolated using Micro Fast Track mRNA Isolation Kit (Invitro gen). Based on 100 ng of mRNA, single-stranded cDNAs were obtained using the First Strand cDNA synthesis Kit (Amersham-Pharmacia). A first PCR was performed to increase specificity using the cDNA as a template, and pSP11-1 (5-ATGAAATCTGCTATTTATGCTITATTATG-3: SEQ ID NO: 44) and NotI-d (T) 18 (Amersham Pharmacia Biotec) as primers. Next, a second PCR was performed using the PCR product as a template, and pSP11-2 (5-TTCATATTCATCGTTTCAAGTC-3: SEQ ID NO: 45) and RT-1 (5-ACTGGAAGAATTCGCGGC-3: SEQ ID NO: 46) as primers. The PCR product was inserted into pCR2.1 vector using TA cloning Kit (Invitrogen), and cloning was performed. Thus, the nucleotide sequence was determined with a DNA sequencer (CEQ2000, Beckman Coulter).

Stigmas were collected from B. oleracea plants having different S-haplotypes, and then the nucleotide sequences of SRK alleles were determined in a manner similar to that described above.

2. Preparation of SP11 Probes for Detecting S-haplotypes

SP11 probes for detecting S-haplotypes were prepared from among the above SP11 genes based on the sequences of regions (regions from which sequences of highly conserved signal peptide portions had been removed: hereinafter described as “SP11*”) that are highly specific to S-haplotypes. Similarly, SRK probes for detecting S-haplotypes were prepared from among the above SRK genes based on the sequences of the regions (regions from which sequences of highly conserved signal peptide portions had been removed: hereinafter described as “SRK*”) that are highly specific to S-haplotypes.

Table 1 describes SEQ ID NOS. of SP11* probes (“SP11* probe”) for detecting each S-haplotype corresponding to SP11 genes identified in 1. Further, table 2 describes SEQ ID NOS. of each SRK* probe corresponding to SRK genes.

TABLE 1

SPl1 gene and SPl1* probe for detecting each S-haplotype
(B. oleracea)

| | | |
|---|---|---|
| | | detectable |
| SPl1 gene | SPl1* probe | S-haplotype |

| | | | | |
|---|---|---|---|---|
| BoSPl1-4 | (SEQ ID NO: 1) | BoSPl1-4* | (SEQ ID NO: 18) | S⁴ |
| BoSPl1-7 | (SEQ ID NO: 2) | BoSPl1-7* | (SEQ ID NO: 19) | S⁷ |
| BoSPl1-8 | (SEQ ID NO: 3) | BoSPl1-8* | (SEQ ID NO: 20) | S⁸ |
| BoSPl1-12 | (SEQ iD NO: 4) | BoSPl1-12* | (SEQ ID NO: 21) | S¹² |
| BoSPl1-14 | (SEQ ID NO: 5) | BoSPl1-14* | (SEQ ID NO: 22) | S¹⁴ |
| BoSPl1-18 | (SEQ ID NO: 6) | BoSPl1-18* | (SEQ ID NO: 23) | S¹⁸ |
| BoSPl1-20 | (SEQ ID NO: 7) | BoSPl1-20* | (SEQ ID NO: 24) | S²⁰ |
| BoSPl1-24 | (SEQ iD NO: 8) | BoSPl1-24* | (SEQ ID NO: 25) | S²⁴ |
| BoSPl1-25 | (SEQ iD NO: 9) | BoSPl1-25* | (SEQ ID NO: 26) | S²⁵ |
| BoSPl1-29 | (SEQ ID NO: 10) | BoSPl1-29* | (SEQ ID NO: 27) | S²⁹ |
| BoSPl1-32 | (SEQ ID NO: 11) | BoSPl1-32* | (SEQ ID NO: 28) | S³² |
| BoSPl1-39 | (SEQ ID NO: 12) | BoSPl1-39* | (SEQ ID NO: 29) | S³⁹ |
| BoSPl1-46 | (SEQ ID NO: 13) | BoSPl1-46* | (SEQ ID NO: 30) | S⁴⁶ |
| BoSPl1-57 | (SEQ ID NO: 14) | BoSPl1-57* | (SEQ ID NO: 31) | S⁵⁷ |
| BoSPl1-58 | (SEQ ID NO: 15) | BoSPl1-58* | (SEQ ID NO: 32) | S⁵⁸ |
| BoSPl1-62 | (SEQ ID NO: 16) | BoSPl1-62* | (SEQ ID NO: 33) | S⁶² |
| BoSPl1-64 | (SEQ ID NO: 17) | BoSPl1-64* | (SEQ ID NO: 34) | S⁶⁴ |

TABLE 2

SRKgene and SRK* probe for detecting each S-haplotype
(B. oleracea and B.rapa)

| | | |
|---|---|---|
| | | detactable |
| SRK gene | SRK* probe | S-haplotype |

| | | | | |
|---|---|---|---|---|
| BoSRK01 | (SEQ ID NO: 47) | BoSRK01* | (SEQ ID NO: 84) | S¹ |
| BoSRK07 | (SEQ ID NO: 48) | BoSRK07* | (SEQ ID NO: 85) | S⁷ |
| BoSRK08 | (SEQ ID NO: 49) | BoSRK08* | (SEQ ID NO: 86) | S⁸ |
| BoSRK11 | (SEQ ID NO: 50) | BoSRK11* | (SEQ ID NO: 87) | S¹¹ |
| BoSRK12 | (SEQ ID NO: 51) | BoSRK2* | (SEQ ID NO: 88) | S¹² |
| BoSRK16 | (SEQ ID NO: 52) | BoSRK16* | (SEQ ID NO: 89) | S¹⁶ |
| BoSRK20 | (SEQ ID NO: 53) | BoSRA20* | (SEQ ID NO: 90) | S²⁰ |
| BoSRK24 | (SEQ ID NO: 54) | BoSRK24* | (SEQ ID NO: 91) | S²⁴ |
| BoSRR25 | (SEQ ID NO: 55) | BoSRK25* | (SEQ ID NO: 92) | S²⁵ |
| BoSRK32 | (SEQ ID NO: 56) | BoSRK32* | (SEQ ID NO: 93) | S³² |
| BoSRK33 | (SEQ ID NO: 57) | BoSRK33* | (SEQ ID NO: 94) | S³³ |
| BoSRK35 | (SEQ ID NO: 58) | BoSRK35* | (SEQ ID NO: 95) | S³⁵ |
| BoSRK36 | (SEQ ID NO: 59) | BoSRK36* | (SEQ ID NO: 96) | S³⁶ |
| BoSRK38 | (SEQ ID NO: 60) | BoSRk38* | (SEQ ID NO: 97) | S³⁸ |
| BoSRK39 | (SEQ ID NO: 61) | BoSRK39* | (SEQ iD NO: 98) | S³⁹ |
| BoSRK45 | (SEQ ID NO: 62) | BoSRK45* | (SEQ ID NO: 99) | S⁴⁵ |
| BoSRK50 | (SEQ ID NO: 63) | BoSRK50* | (SEQ ID NO: 100) | S⁵⁰ |
| BoSRK51 | (SEQ ID NO: 64) | BoSRK51* | (SEQ ID NO: 101) | S⁵¹ |
| BoSRK57 | (SEQ ID NO: 65) | BOSRK57* | (SEQ ID NO: 102) | S⁵⁷ |
| BoSRK58 | (SEQ ID NO: 66) | BoSRK58* | (SEQ ID NO: 103) | S⁵⁸ |
| BoSRK62 | (SEQ ID NO: 67) | BoSRK62* | (SEQ ID NO: 104) | S⁶² |
| BoSRK64 | (SEQ ID NO: 68) | BoSRK64* | (SEQ ID NO: 105) | S⁶⁴ |
| BrSRK65 | (SEQ ID NO: 69) | BrSRK65* | (SEQ ID NO: 106) | S⁶⁵ |
| BrSRK26 | (SEQ ID NO: 70) | BrSRK26* | (SEQ ID NO: 107) | S²⁶ |
| BrSRA27 | (SEQ ID NO: 71) | BrSRK27* | (SEQ ID NO: 108) | S²⁷ |
| BrSRK30 | (SEQ ID NO: 72) | BrSRK30* | (SEQ ID NO: 109) | S³⁰ |
| BrSRK32 | (SEQ ID NO: 73) | BrSRK32* | (SEQ ID NO: 110) | S³² |
| BrSRK33 | (SEQ ID NO: 74) | BrSRA33* | (SEQ ID NO: 111) | S³³ |
| BrSRK34 | (SEQ ID NO: 75) | BrSRK34* | (SEQ ID NO: 112) | S³⁴ |
| BrSRK35 | (SEQ ID NO: 76) | BrSRK35* | (SEQ ID NO: 113) | S³⁵ |
| BrSRK36 | (SEQ ID NO: 77) | BrSRK36* | (SEQ ID NO: 114) | S³⁶ |
| BrSRK37 | (SEQ ID NO: 78) | BrSRK37* | (SEQ ID NO: 115) | S³⁷ |
| BrSRK41 | (SEQ ID NO: 79) | BrSRK41* | (SEQ ID NO: 116) | S⁴¹ |
| BrSRK47 | (SEQ ID NO: 80) | BrSRK47* | (SEQ ID NO: 117) | S⁴⁷ |
| BrSRK48 | (SEQ ID NO: 81) | BrSRK48* | (SEQ ID NO: 118) | S⁴⁸ |
| BrSRK49 | (SEQ ID NO: 82) | BrSRK49* | (SEQ ID NO: 119) | S⁴⁹ |
| BrSRK99 | (SEQ ID NO: 83) | BrSRK99* | (SEQ ID NO: 120) | S⁹⁹ |

### EXAMPLE 2

Identification of S-haplotypes in Genomic DNA by Dot Blotting

Among S tester lines (lines differing in S-haplotypes) of B. oleracea preserved at Tohoku University, Graduate School of Agricultural Science, Laboratory of Plant Breeding and Genetics, 16 lines: S⁵, S⁶, S⁷, S⁸, S⁹, S¹², S¹³, S¹⁴, S¹⁵, S²⁵, S²⁹, S³², S⁵⁷, S⁵⁸, S⁶⁰, and S⁶⁵, and 16 plants selected from selfed progeny of S³⁹×broccoli of B. oleracea were used.

1. Preparation of Genomic DNA

Isolation of genomic DNA was performed by the CTAB (Cetyl trimethyl ammonium bromide) method with modified processes of crushing and extraction. 3 g of leaves of the above B. oleracea was frozen and crushed with liquid nitrogen, and then DNA was extracted with a mixed solution of 6 ml of 2×CTAB solution (2% CTAB, 100 mM Tris-HCl pH 8.0, 1.4 M NaCl, and 20 mM EDTA), 3 ml of 1×CTAB solution, and 0.5 ml of 10% CTAB solution. Protein was removed with chloroform·isoamylalcohol (24:1), and then CTAB precipitation buffer (1% CTAB, 50 mM Tris-HCl pH8.0, and 1 mM EDTA) was added, so that CTAB-DNA was precipitated. The precipitated CTAB-DNA was spun, and then dissolved in NaCl-TE (1 M NaCl, 10 mM Tris-HCl pH8.0, 1 mM EDTA). Isopropanol was added to the solution, and then the precipitated DNA was washed with ethanol. The DNA was air-dried, and then dissolved in 1×TE, so as to perform RNase treatment.

Since the uniformity of the DNA concentration is important, concentration was measured by ethidium bromide staining after electrophoresis, while measurement was also performed using a DQ200 DyNA Quant TM 200 Fluorometer (Pharmacia).

2. Blotting of DNA Samples

1 μg, 2 μg, and 5 μg of the genomic DNAs of each line were respectively denatured into single strands by alkali denaturation and thermal denaturation, dot-blotted to a membrane (Nytran N: Schleicher & Schuell), and then subjected to neutralization treatment. The product was exposed to ultraviolet radiation using GS GENE LINKER (BIO-RAD), and then baked at 80° C. for 1 hour.

3. Labeling of DNA Samples

Next, PCR reaction was performed using as a template a plasmid DNA having SP11 cDNA (S⁸, S¹² and S⁵⁷ haplotypes) inserted therein, and primers for amplifying SP11, and digoxigenin (DIG)-labeled dNTP added as a substrate, thereby performing DIG-labeling. PCR reaction was performed with 0.1×SSC containing 0.1% SDS at 68° C.

The genomic DNA of each line was dot-blotted on a membrane, and then DNAs of S⁷, S¹², and S³² haplotypes were detected using SP11* probes prepared in Example 1.

4. Results

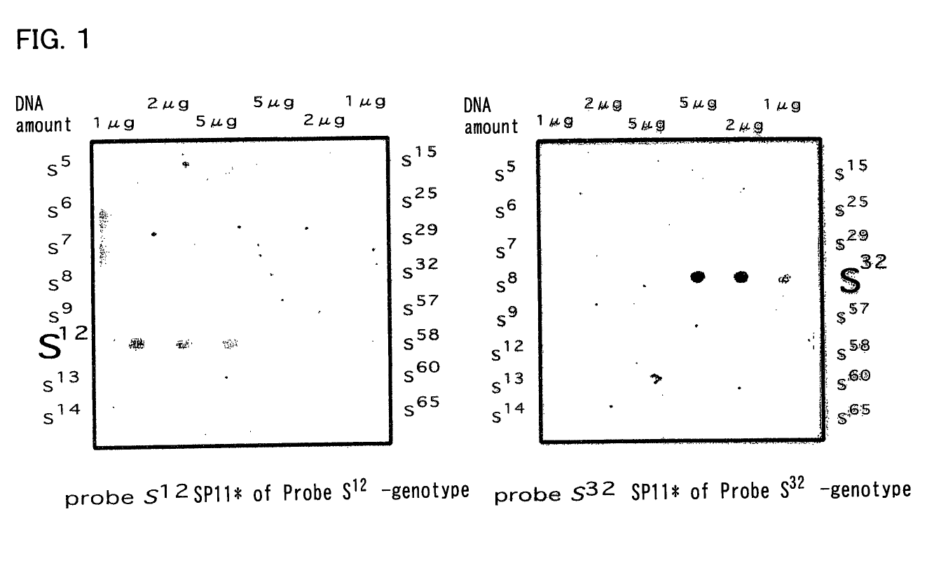

When SP11* probes of a group of plants having S⁷-haplotype were used, signal was detected for plants of S⁷ and S⁹-haplotypes, when SP11* probes of a group of plants having S¹²-haplotype were used, signal was detected only for plants having S¹²-haplotype, and when SP11* probes of a group of plants having S³²-haplotype were used, signal was detected only for plants having S³²-haplotype (FIG. 1). Plants of S⁹-haplotype detected with the above SP11* probes of S⁷-haplotype were shown by later analysis to be of S⁷-haplotype, suggesting that this method is also useful in the detection of contamination of lines.

### EXAMPLE 3

Identification of S-haplotypes by Dot Blotting Probes

Unlabeled SP11* of each S-haplotype was dot-blotted with a 10-fold concentration gradient onto a membrane. Probes used herein were labeled with digoxigenin (DIG) by the following two methods using genomic DNA as a template.

1. Labeling Using Double-sided Primer:

DIG-labeling was performed using a genomic DNA as a template, and primers for amplifying SP11* of a group of plants having S⁸-haplotype. PCR was performed for 30 cycles, each cycle consisting of 93° C. for 30 seconds, 55° C. for 30 seconds, and 72° C. for 30 seconds.

2. Labeling Using Single-sided Primer:

DIG labeling was performed using a genomic DNA as a template, and SP11-A-F. A PCR reaction condition of 93° C. for 30 seconds, 55° C. for 30 seconds, and 72° C. for 10 seconds was performed for 80 cycles. Primers used herein are shown in Table 3.

TABLE 3

Primers for labeling SPl1 (B. oleracea)

Detectable S-haplotype

Forward primer

| | | | |
|---|---|---|---|
| SPl1-A-F | 5′-AAGTGGAAGCTAAT-3′ | SEQ ID NO: 35 | 6, 7, 9, 11, 12, 13, 14, 18, 20, 2 |
| | | | 4, 25, 29, 32, 46, 57, 58, 63 |
| SPl1-B-F | 5′-GAAGTGGAAGCT-3′ | SEQ ID NO: 36 | 4, 39, 60, 62, 64 |
| SPl1-C-F | 5′-TTGCCTGGACGTTGTCGC-3′ | SEQ ID NO: 37 | 8, 62 |

Reverse primer

| | | | |
|---|---|---|---|
| SPl1-A-R | 5′-TTGTAGTTGTCAACTA-3′ | SEQ ID NO: 38 | 4, 11, 18, 25, 46, 60, 63 |
| SPl1-B-R | 5′-TGGACGATGTGATTGT-3′ | SEQ ID NO: 39 | 9, 14, 39, 57, 64 |
| SPl1-C-R | 5′-TTGTGAATGTAAAT-3′ | SEQ ID NO: 40 | 7, 12, 13, 24, 32, 62 |
| SPl1-D-R | 5′-GAAGACGAAGCCTCTTAATTGC-3′ | SEQ ID NO: 41 | 6, 8, 9 |
| SPl1-E-R | 5′-AAAGGACGATGTTGTT-3′ | SEQ ID NO: 42 | 14, 20, 58 |
| SPl1-F-R | 5′-ATAAAAGGGGACATTGT-3′ | SEQ ID NO: 43 | 18, 29 |

In the case of PCR labeling, a combination of a forward primer and a reverse primer is used. All the primers may be mixed.

Further, in the case of PCR labeling with single-sided primers, either a forward primer or reverse primer is used. Forward primers or reverse primers may be mixed, respectively.

Detection was performed in a manner similar to Example 2.

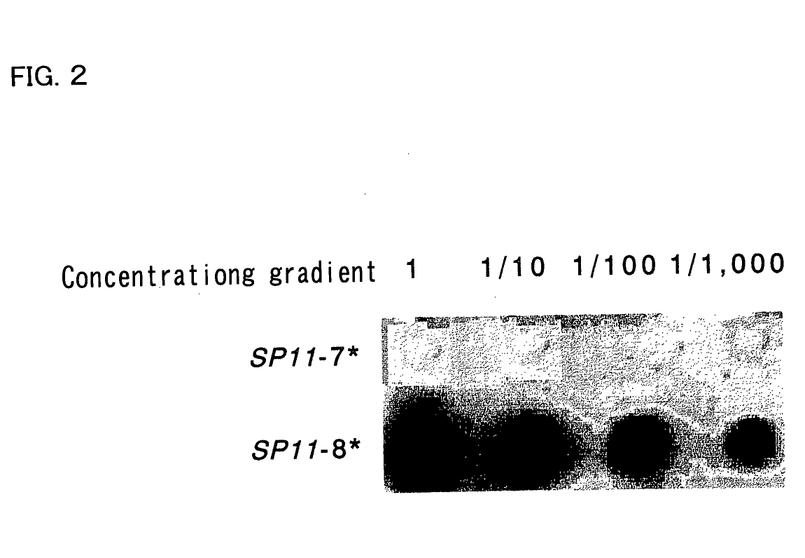

SP11* probes of a group of plants having S⁷ and S⁸-haplotypes were dot-blotted onto a membrane, and then DIG-labeled by the PCR method using primers for amplifying the SP11*region of a group of plants having S⁸-haplotype, and the genomic DNA of the group of plants having S⁸-haplotype as a template. When detection was performed using the probe, signal was detected only for SP11* probes of the group of plants having S⁸-haplotype (FIG. 2).

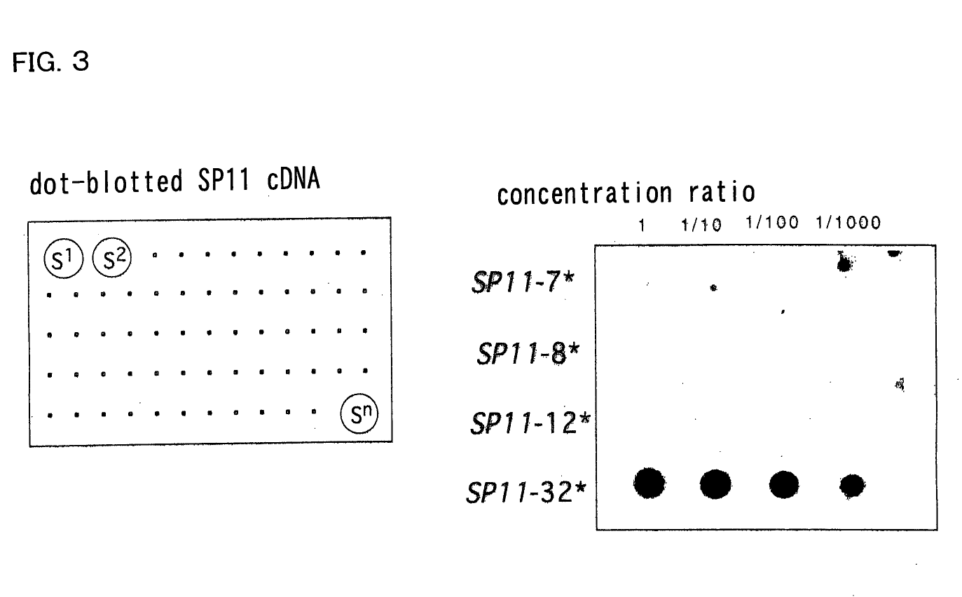

Also in the case of a labeling method using single-sided primers, when SP11* probes of a group of plants having S⁷-, S⁸-, S¹²-, and S³²-haplotypes were dot-blotted with a concentration gradient onto a membrane, and DIG-labeled using S³² genomic DNA as a template, and then detection was performed using the probe, signal was detected only for SP11* probes of the group of plants having S³²-haplotype (FIG. 3).

3. Results

As described above, it was shown that both the method, which labels with several types of single-sided primers using a genomic DNA as a template, and the method, which labels with several types of double-sided primers using a genomic DNA as a template, can be utilized. Since labeling efficiency was better in the case of double-sided primers, and signal was weak in the case of the method, which labels with single-sided primers, it was considered that the use of double-sided primers is better. However, single-sided primers are advantageous in that, many S-haplotypes could already be labeled with only the single-sided primers designed this time, and a small number of the primers is sufficient for mixing (when mixing is necessary). At the same time, double-sided primers can be problematic, such that designing of the counterpart primers is difficult, and that a large number of primers to be mixed is required.

### EXAMPLE 4

Identification of S-haplotypes by Southern Blotting of Genomic DNA

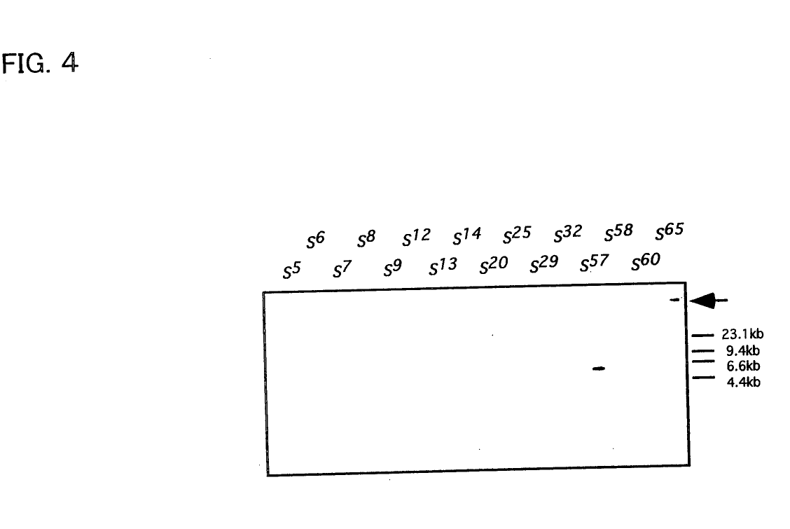

Southern blotting was performed for 16 S tester lines of B. oleracea that had been used in Example 2 using as a probe (SP11-57 probe) SP11 cDNA sequence (BoSP11-57 (SEQ ID NO: 14) in Table 1) of S⁵⁷ haplotype. The result is shown in FIG. 4.

As is clear from FIG. 4, the SP11-57 probe specifically reacted with the tester line (having homozygous S⁵⁷ haplotype) of S⁵⁷ haplotype.

### EXAMPLE 5

Comparison of S-haplotype Identification (Dot Blot Method) Using SP11* probe and that using SP11 probe

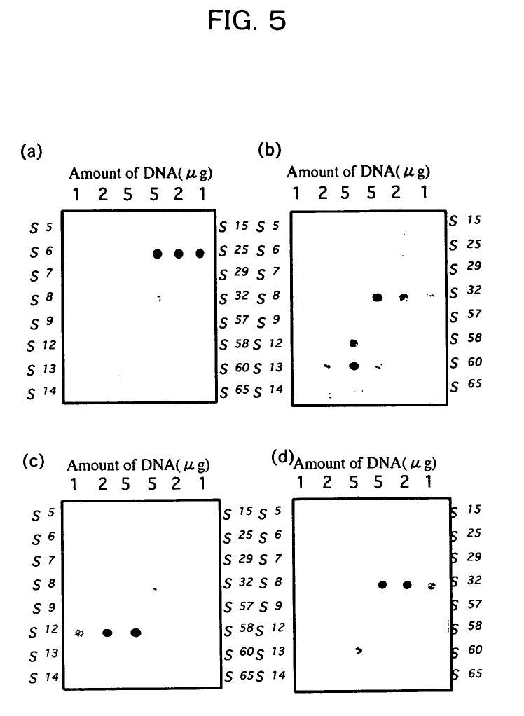

An SP11 probe that contains the sequence of a signal peptide portion and an SP11* probe that does not contain the sequence of the signal peptide portion were compared and studied for their specificity. The test was performed, according to Example 2, on 16 S tester lines of B. oleracea by the dot blot method using as probes (a) an SP11-25 probe, (b) an SP11-32 probe, (c) an SP11-12* probe, and (d) an SP11-32* probe. The results are shown in FIG. 5.

As is clear from FIG. 5, the SP11-25 probe specifically and strongly reacted with S²⁵ plant, but the SP11-32 probe showed cross-reactivity not only with S³² but also with plants of S-haplotypes (other than S³²) at a low concentration. Meanwhile, the SP11-12* probe and SP11-32* probe specifically and strongly reacted with plants of S-haplotypes that each of the probes recognizes. In addition, SP11-32* was observed to react also with S¹³-haplotype plant at a high concentration. However, such a degree of reaction was not considered to be a practical problem. Thus, it was confirmed that S-haplotypes can be detected more specifically by the use of an SP11* probe, from which the signal peptide region had been removed.

### EXAMPLE 6

Purity Verification of Parent Line Using SP11* probe

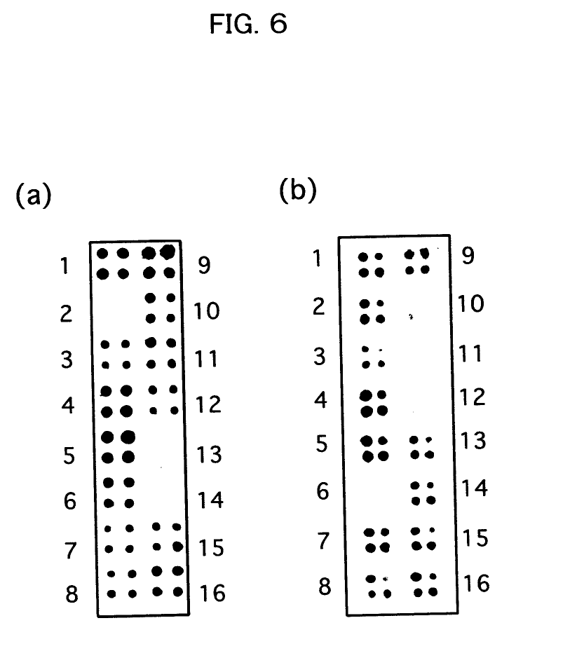

Among homozygous seeds collected, there may be heterozygous seeds resulting from the contamination with pollens. Hence, whether or not dot blotting can be used for purity verification was examined using F₂ plants. The test was performed on F₂ generation produced by crossing B. oleracea homozygous plants of S¹⁸ haplotype and S³⁹ haplotype by the dot blot method using (a) an SP11-18* probe and (b) an SP11-39* probe. The results are shown in FIG. 6.

As a result, it was confirmed that signals appeared strongly in homozygous plants and signals appeared weakly in heterozygous plants. Specifically, it was demonstrated that the S-haplotype identification method using an SP11* probe can also be used to verify the seed purity of parent lines and F₁ hybrid cultivars.

### EXAMPLE 7

Distinguishing between S⁷ Homozygous Plants and S¹⁸/S³⁹ Heterozygous Plants Using SP11 * Probe

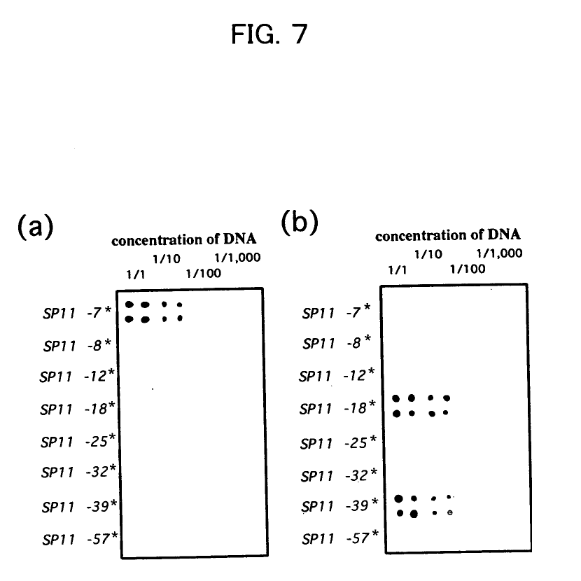

SP11* probes were respectively diluted to 1/1, 1/10, 1/100 and 1/1000, and then dot-blotted onto nylon membranes. Meanwhile, the genomic DNAs of B. oleracea S⁷ homozygous plants and S¹⁸/S³⁹ heterozygous plants extracted by CTAB method were DIG-labeled by PCR. Then, the labeled DNAs were allowed to react with the above dot-blotted probes, so that detection was performed. The results are shown in FIG. 7 ((a) S⁷ homozygous plant, and (b) S¹⁸/S³⁹ heterozygous plant).

As is clear from FIG. 7, the samples of S⁷ homozygous plants specifically reacted only with the SP11-7* probe, and S⁸/S³⁹ heterozygous plants specifically reacted with the SP11-18* probe and the SP11-39* probe.

### EXAMPLE 8

Distinguishing between S¹⁸ Homozygous Plants and S¹⁵ Homozygous Plants Using SP11* Probe

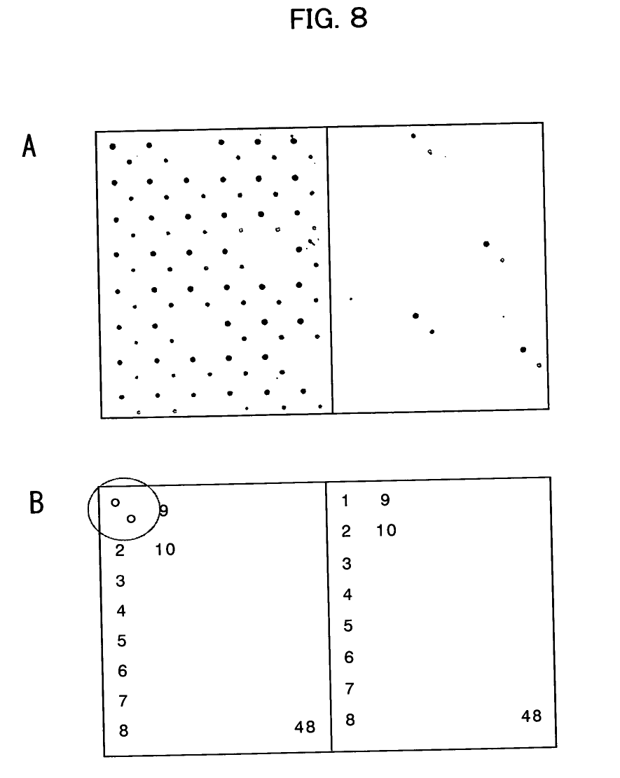

The genomic DNAs of B. oleracea S¹⁸ homozygous plants and S¹⁵ homozygous plants (48 plants in total) extracted by CTAB method were dot-blotted randomly, and then detection was performed using an SP11-18* probe and an SP11-15* probe. FIG. 8(B) schematically shows the blot method. Dot blotting was repeated twice per plant, and the upper left dot contains double volume of DNAs in the lower right dot. Numerals respectively denote each plant number. The results are shown in FIG. 8(A).

As a result of the above dot blot analysis, plant Nos. 17, 22, 36, and 47 were confirmed to be S¹⁵/S¹⁵ plants, and others were confirmed to be S¹⁸/S¹⁸ plants.

### EXAMPLE 9

Identification of S-haplotypes of Raphanus sativus

According to the method of Example 1, S-haplotype specific SP11 of Raphanus sativus (radish) was specified. Specifically, anthers were collected from Raphanus sativus plants of different S-haplotypes, and the mRNA was isolated using a Micro Fast Track mRNA Isolation Kit (Invitrogen). Next, based on this mRNA, a single stranded cDNA was prepared using the First Strand cDNA synthesis Kit (Amersham-Pharmacia). A first PCR was performed using the cDNA as a template, and pSP11-1 (5′-ATGAAATCTGCTATTTATGCTTTATTATG-3′: SEQ ID NO: 44) and NotI-d(T) 18 (Amersham Pharmacia Biotec) as primers. Further, using the PCR product as a template and pSP11-2 (5′-TTCATATTCATCGTTTCAAGTC-3′: SEQ ID NO: 45) and RT-1 (5′-ACTGGAAGAATTCGCGGC-3′: SEQ ID NO: 46) as primers, a second PCR was performed. The PCR product was inserted into pCR2.1 vector using TA cloning Kit (Invitrogen), cloning was performed, and then the nucleotide sequences were determined using a DNA sequencer (CEQ2000, Beckman Coulter).

The thus determined Raphanus sativus SP11 genes and specific SP11* probes (SP11 genes that do not contain signal peptide regions) for detecting the genes are listed in Table 4 below.

TABLE 4

SPl1gene and SFl1* probes for detecting each S-haplotype
(Raphanus sativus)

| | | |
|---|---|---|
| | | detectable |
| SPl1gene | SPl1* probe | S-haplotype |

| | | | | |
|---|---|---|---|---|
| RaSPl1-1 | (SEQ ID NO: 121) | RaSPl1-1* | (SEQ ID NO: 128) | S¹ |
| RaSPl1-2 | (SEQ ID NO: 122) | RaSPl1-2* | (SEQ ID NO: 129) | S² |
| RaSPl1-4 | (SEQ ID NO: 123) | RaSPl1-4* | (SEQ ID NO: 130) | S⁴ |
| RaSPl1-6 | (SEQ ID NO: 124) | RaSPl1-6* | (SEQ ID NO: 131) | S⁶ |
| RaSPl1-13 | (SEQ ID NO: 125) | RaSPl1-13* | (SEQ ID NO: 132) | S¹³ |
| RaSPl1-20 | (SEQ ID NO: 126) | RaSPl1-20* | (SEQ ID NO: 133) | S²⁰ |
| RaSPl1-21 | (SEQ ID NO: 127) | RaSPl1-21* | (SEQ ID NO: 134) | S²¹ |

### EXAMPLE 10

Identification of S-haplotypes by Dot Blotting Raphanus sativus

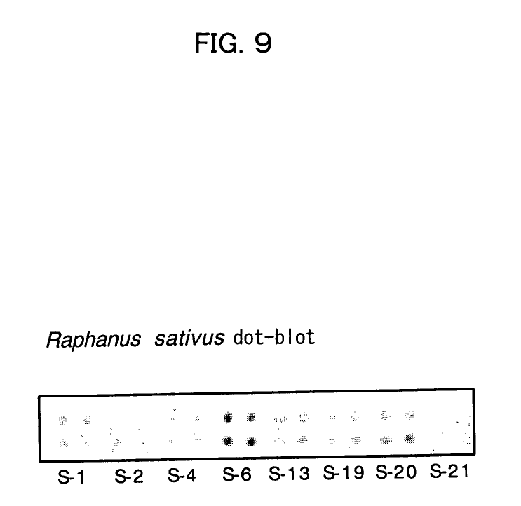

The genomic DNAs of Raphanus sativus plants having S-haplotypes of each S¹, S², S⁴, S⁶, S¹³, S¹⁹, S²⁰, or S²¹ were isolated by CTAB method according to the method of Example 2. Dot blotting was performed using the thus-prepared genomic DNAs and a DIG-labeled Raphanus sativus SP11-6* probe. The result is shown in FIG. 9.

As is clear from FIG. 9, the tester line of S⁶ haplotype specifically and strongly reacted with the SP11-6* probe.

According to the present invention, S-haplotypes of plants belonging to Brassicaceae can be rapidly and simply identified.

All publications, patents and patent applications cited herein are incorporated herein by reference in their entirety. Sequence Listing Free Text SEQ ID NOS: 18 to 34: probes specific to S-haplotypes of Brassicaceae SEQ ID NOS: 35 to 46: Primers SEQ ID NOS: 84 to 120: probes specific to S-haplotypes of Brassicaceae SEQ ID NOS: 128 to 134: probes specific to S-haplotypes of Brassicaceae

What is claimed is:

1. A DNA fragment, which consists of a nucleotide sequence contained in at least a gene selected from the group consisting of an S-locus cysteine-rich protein gene, an S-locus receptor kinase gene and an S-locus glycoprotein gene that are present on the S-locus of plants belonging to Brassicaceae, and with which S-haplotypes can be specified.

2. A DNA fragment, which consists of a nucleotide sequence contained in an S-locus cysteine-rich protein gene, and/or an S-locus receptor kinase gene that is present on the S-locus of plants belonging to Brassicaceae and with which S-haplotypes can be specified.

3. A DNA fragment, which is defined by any one of the sequences selected from the group consisting of SEQ ID NOS: 1 to 17, SEQ ID NOS: 47 to 83, and SEQ ID NOS: 121 to 127, and with which S-haplotypes can be specified.

4. A method for identifying S-haplotypes, which comprises detecting DNA fragments with which S-haplotypes can be specified from an plant or a group of plants belonging to Brassicaceae

5. The method according to claim 4, wherein the DNA fragment, with which S-haplotypes can be specified consists of a nucleotide sequence contained in at least a gene selected from the group consisting of an S-locus cysteine-rich protein gene, an S-locus receptor kinase gene and an S-locus glycoprotein gene that are present on the S-locus of plants belonging to Brassicaceae.

6. The method according to claim 4, wherein the DNA fragment, with which S-haplotypes can be specified, consists of a nucleotide sequence contained in an S-locus cysteine-rich protein gene and/or an S-locus receptor kinase gene that are present on the S-locus of plants belonging to Brassicaceae.

7. The method according to claim 4, wherein the DNA fragment, with which S-haplotypes can be specified, is defined by any one of the sequences selected from the group consisting of SEQ ID NOS: 1 to 17, SEQ ID NOS: 47 to 83, and SEQ ID NOS: 121 to 127.

8. A method of purity verification or quality control for seeds, which uses the method of claim 5.

9. A method for plant breeding, which uses the method of claim 5.

10. A plant or a cultivar, which is produced by the breeding method of claim 9.

11. An oligonucleotide primer, which is for specifically amplifying the DNA fragment of claim 1, and has sequential 10 to 50 nucleotides in length.

12. A probe, which hybridizes specifically to the DNA fragment of claim 1, and is for detecting the fragment.

13. A probe, which is for detecting an S-haplotype specific DNA fragment which is defined by any one of the sequences selected from the group consisting of SEQ ID NOS: 18 to 34, SEQ ID NOS: 84 to 120, and SEQ ID NOS: 128 to 134.

14. The method according to claim 5, which is performed using a probe defined by any one of the sequences selected from the group consisting of SEQ ID NOS: 18 to 34, SEQ ID NOS: 84 to 120, and SEQ ID NOS: 128 to 134.

15. A method for identifying S-haplotypes, which comprises the following steps of:
1) extracting DNA samples from a plant,
2) immobilizing the above DNA samples onto a support,
3) labeling probes for detecting S-haplotype specific DNA fragments, and hybridizing the probes to the above DNA samples, and
4) identifying the S-haplotypes of the plant based on the above labels.

16. A method for identifying S-haplotypes, which comprises the following steps of:
1) immobilizing probes for detecting S-haplotype specific DNA fragments onto a support,
2) extracting and labeling DNA samples from a plant,
3) hybridizing the above labeled DNA samples to the probes on the support, and
4) identifying the S-haplotypes of the plant based on the above labels.

17. A support for identifying S-haplotypes, on which the probe of claim 12 is immobilized.

18. A support for identifying the S-haplotypes, on which the probe of claim 13 is immobilized.

19. The support for identifying the S-haplotypes of claim 17, wherein any one support selected from the group consisting of a membrane, a glass plate, a capillary and a bead is used as the support.

20. A kit for identifying the S-haplotypes, which comprises at least one or more elements selected from the following 1) to 3):
1) an oligonucleotide primer, which is for specifically amplifying the DNA fragment of claim 1, and has sequential 10 to 50 nucleotides in length,
2) a probe, which hybridizes specifically to the DNA fragment of claim 1, and is for detecting the fragment; and
3) a support, which has probes immobilized thereon that specifically hybridize to the DNA fragments of claim 1, and is for detecting the fragments.
